SEQUENCE LISTING (2 sequences)

SEQ ID NO: 1 - protein, 34 aa, synthetic construct
GNYNYLYRLFRKSNLKPFERDISTEIYQAGSTPC

SEQ ID NO: 2 - protein, 22 aa, synthetic construct
CYFPLQSYGFQPTNGVGYQPYR

(19) United States
(12) Patent Application Publication
(10) Pub. No.: US 2025/0295606 A1
(43) Pub. Date: Sep. 25, 2025

(54) BIOSOLUBLE POLYMER OR PARTICLE FOR DELIVERY OF AN ACTIVE AGENT AND A METHOD FOR THE PRODUCTION

(71) Applicant: Tijani Holding B.V., Eindhoven (NL)

(72) Inventor: Amina TIJANI, Oss (NL)

(21) Appl. No.: 18/702,644

(22) PCT Filed: Oct. 19, 2022
(86) PCT No.: PCT/NL2022/050593

§ 371 Date: Apr. 18, 2024

(30) Foreign Application Priority Data
Oct. 19, 2021 (EP) 21203511.7

(51) Int. Cl. A61K 9/51; A61K 9/00; A61K 38/28; A61P 3/10

(52) U.S. Cl. CPC A61K 9/5192; A61K 9/006; A61K 38/28; A61P 3/10

(57) ABSTRACT

The present invention refers to a method for producing a polymer in form of a gel or a particle, and to the resulting polymer, gel and particle, respectively. The polymer comprises a carbon donor and a metal oxide precursor, a metal oxide or a combination thereof and optionally an active agent. The invention is further directed to a composition and film comprising such polymer, and their use as a medicament for example in treating diabetes, obesity, neuronal disease, viral infection or cancer.

### CROSS-REFERENCE TO RELATED PATENT APPLICATIONS

This application is a U.S. National Stage Application under 35 U.S.C. § 371 of International Patent Application No. PCT/NL2022/050593, filed Oct. 19, 2022, which claims the benefit of priority of European Patent Application number 21203511.7 filed Oct. 19, 2021, both of which are incorporated by reference in their entireties. The International Application was published on Apr. 27, 2023, as International Publication No. WO/2023/068928.

The instant application contains a Sequence Listing which has been submitted electronically in XML format and is hereby incorporated by reference in its entirety. Said XML copy, created on Apr. 18, 2024, is named AS FILED-P131411US00-Sequence Listing-10114/012481-USO and is 2.78 kilobytes in size.

The present invention refers to a method for producing a polymer in form of a gel or a particle, and the resulting polymer, gel and particle, respectively. The invention is further directed to a composition and film comprising such polymer, and their use as a medicament for example in treating diabetes, a neuronal disease, a viral infection or cancer.

### TECHNICAL BACKGROUND

Particles are an important tool for delivery of all kinds of active agents and have a wide area of application. Advantageously, particles should successfully protect the active agent until it reaches its final target, but at the same time should be completely biosoluble to avoid any undesired side effects in the environment such as a body.

Biosoluble polymers and particles, respectively, represent a class of polymers and particles that can be gradually broken down by a specific activity for example enzymatic activity resulting in natural products such as gases, water, biomass, organic and inorganic salts. Hence, biosoluble polymers and particles exhibit great potential in diverse fields of technology and applications.

Biosoluble particles consist for example of (bio)polymers formed by a polymerization reaction of monomers. Such polymerization reactions allow the formation of polymers in diverse structures like chains, sheets, particulates or complex three-dimensional networks. Due to their ability to form complex three-dimensional structures biosoluble polymers are able to encapsulate active agent(s).

Active agents, especially drugs, are often instable, insoluble and/or toxic limiting their desired effect. Thus, it is well known in the pharmaceutical field to use delivery systems in form of particles, especially hollow, core-shell, porous or non-porous nanoparticles, for example known as “vectors”, for the encapsulation and/or immobilization of active agents. Such particles may protect the active agent from degradation, deactivation, complexation with other entities, early release, promote solubility in certain biological environments allowing better absorption of the active agent and preserving its therapeutic effect. The use of particles improves the active agent's bioavailability, and its controlled release at the desired site of action. Decorating the surface of a nanoparticle with molecular recognition elements may result in improved cell targeting and bioavailability of the encapsulated active agent.

The production of nanoparticles based on natural polymers is for example described in WO 2009/081287. Even if such nanoparticles have the advantage of being non-toxic and biodegradable, the method of obtaining them is laborious, particularly due to the need to cultivate the microorganisms producing such polysaccharides, and due to the separation and purification stages of the natural nanoparticles obtained in this manner.

Apart from their known advantages, drug delivery systems based on organic polymers have shown several disadvantages and limitations including: (1) in vivo instability of active targeted drug delivery systems, (2) some immune reactions may occur against intravenous administered carrier systems, (3) requirement of highly sophisticated technology for the formulation, (4) difficulty to maintain stability of dosage formulations, (5) low drug load, and (6) drug release can occur earlier before approaching the target disease site (Dikmen et al., 2011).

Immune reactions such as complement activation-related pseud-oallergy (CARPA) are triggered for example by polyethylene glycol (PEG) after intravenous administration. PEG can trigger complement activation by enhancing fluid phase complement turnover and a MASP-2-regulated process in concentration and M_{wt}-dependent manner (Hamad I. et al., Molecular Immunology 46, (2008), 225-232).

Hollow inorganic nanoparticles are easier to obtain. Most widespread are nanoparticles of silica, a trace element that is well absorbed and assimilated by the human body, and not toxic if it is not inhaled. Usually, silica nanoparticles are obtained by using a silicon alkoxide such as tetra-ethyl-ortho-silicate (TEOS) or tetra-methyl-ortho-silicate (TMOS). As an example, document WO99/36357 describes the obtaining of mesoporous silica nanoparticles by the polycondensation of a metal alkoxide in the presence of a blowing agent which can be a carbohydrate.

Micro- and nanoscale particles are often based on metal-oxide polymers which are produced by sol-gel technique. Sol-gel technique represents a low-temperature method using chemical precursors. It enables researches to design and fabricate a wide variety of different materials comprising monolithic and porous glasses, fibers, powders, thin film, nanocrystallites, photonic crystals etc. with unique chemical and physical properties. Sol-gel materials are for example based on silica, alumina, titanium and other compounds.

WO2008/062426 for example refers to a controlled delivery and release formulation for oral administration comprising galanthamine. WO2009/136992 discloses a polymer-based pharmaceutical composition containing exenatide for oral or rectal administration. Further, WO2014/118774 provides a silica-based pharmaceutical composition for oral use comprising at least two bioactive proteins associated with glucose metabolism. None of these documents discloses however particles consisting of biosoluble polymers of covalently connected metal oxide and a carbon originating for example from a carbohydrate.

Prior art polymers and particles, respectively, containing active agents often have the problem of deficient loading or deficient concentration of the encapsulated active agent, complexity of the formulation, and/or the inability to deliver or release a sufficient amount of active agent at the site of action. Therefore, there is a need for polymers and particles that reliably provide a substantial dose of the active agent which is delivered controlled in a slow and fast mode, respectively, at the site of action depending on the requirements.

Moreover, active agents are protected from degradation. In particular, proteins and peptides or nucleic acids are sensitive to degradation for example enzymatic degradation or degradation due to other conditions such as high temperature or a basic or an acidic pH. However, there is an enormous need for oral administration of pH and/or heat sensitive drugs such as insulin, incretin and their analogues to avoid administration via injection. The present invention provides the great advantage of mucosal resorption of such active agents for example via oral such as sublingual or buccal administration.

Another great advantage is the use of the polymer, particle, composition or film of the present invention in the field of vaccination. The vaccine is comprised by the polymer and particle, respectively, which protects the vaccine from degradation. The present invention optionally acts as an adjuvant and replaces other adjuvants or is combined with other adjuvants such as KLH. The polymer, particle, composition or film of the present invention comprising a vaccine is preferably administered via injection. Successful vaccination requires reliable, complete intake of the vaccine in the organism to stimulate the desired immune response. It results in a high immune response, e.g., represented by a high antibody titer, without causing undesired side effects or only a significantly reduced amount and/or severity of side effects such as thrombosis, dizziness, nausea, fatigue, fever, muscle pain or any other typical side effect of a vaccination.

The present invention is for example used in vaccination for preventing and/or treating a respiratory disease such as Covid, e.g., Covid-19. The particle of the present invention is for example used as a vaccine based on Covid mRNA and Covid Spike administered via the sublingual mucosa of large animal model.

Alternatively, the present invention is used in brain (e.g., CNS) diseases. The development of new drugs for the brain has progressed at a much slower pace than that for the rest of the body. This slow progress has been due in large part to the inability of most drugs to cross the brain capillary wall, which forms the blood-brain barrier (BBB), to enter the brain. Approximately 100% of large-molecule drugs, and greater than 98% of small-molecule drugs do not cross the BBB. Only a small class of drugs, small molecules with a high lipid solubility and a molecular mass of less than 400-500 daltons actually cross the BBB and of the small molecules that cross the BBB, only a small percentage cross the BBB in a pharmaceutically significant amount (Pardridge, Molecular Innovations 3:90-103 2003)). Only a few diseases of the brain respond to the small molecule drugs that can cross the BBB, such as depression, affective disorders, chronic pain and epilepsy. Far more diseases of the brain do not respond to the convention lipid-soluble small molecular mass drugs, such as Alzheimer disease, stroke/neuroprotection, brain and spinal cord injury, brain cancer, HIV infection of the brain, various ataxia-producing disorders, amyotrophic lateral sclerosis (ALS), Huntington disease, childhood inborn genetic errors affecting the brain, Parkinson's disease and multiple sclerosis. Particularly difficult to treat are cancers of the brain. The common forms of cancer in the brain are glioblastoma multiform (GBM) and anaplastic astrocytoma (AA). The mean survival for patients with GBM is approximately 10 to 12 months, while the median survival for patients with AA is 3 to 4 years (Kufe et al. Cancer Medicine, chap 23 and 83, (6^{th} ed. B C Decker, 2003). More cases where treatment of GBM is by surgery and local irradiation result in relapse within 2 to 4 cm of the original tumor margins (Tan A.C. et al., CA CANCER J CLIN 2020; 70:299-312).

The present invention further allows the use of particles in personalized medicine, wherein the active agent is preferably resorbed via the mucosa.

The particle of the present invention is for example basis for compositions or film which comprise a particle of the present invention and overcome the disadvantages of the prior art.

The polymer and particle, respectively, of the present invention is producible in a simplified, economical way. The method for its production is easy to implement and to scale-up. The polymer or particle is adaptable for fast and slow release of an active agent. The active agent is in a stadium of reduced activity and is fully reactivated at the target such as a target cell, tissue and/or organ, or on the field in case of agricultural use. The stadium of reduced activity allows a high concentration of the active agent in the polymer or particle.

### SUMMARY OF THE INVENTION

The present invention refers to a method for the production of a polymer comprising the steps:
a) preparing a saturated solution of a carbon donor such as a carbohydrate, the carbon donor is dissolved in a water/alcohol solvent or water/alcohol/organic solvent wherein the ratio of water:alcohol is from 20:80 to 80:20 or wherein the ratio of water:alcohol:organic solvent is from 5:80:15 to 80:15:5,
b) mixing the saturated solution of step a) with a metal oxide precursor, a metal oxide or a combination thereof,
c) adding an alcoholic or hydro-alcoholic solution of a polycondensation catalyst to step a) and/or step b),
wherein all the steps are performed at a temperature in a range of about −20° C. to about 55° C., preferably about −5° C. to about 25° C. and the carbon donor and the metal oxide precursor, the metal oxide or a combination thereof form a gel;
d) optionally additives are added in step a), b) or c).

Alternatively, it relates to a method for the production of a polymer comprising the steps:
a) preparing a saturated solution of a carbon donor such as a carbohydrate, the carbon donor is dissolved in a water/alcohol solvent or water/alcohol/organic solvent wherein the ratio of water:alcohol is from 20:80 to 80:20 or wherein the ratio of water:alcohol:organic solvent is from 5:80:15 to 80:15:5,
b) mixing the saturated solution of step a) with a metal oxide precursor, a metal oxide or a combination thereof,
c) adding an alcoholic or hydro-alcoholic solution of a polycondensation catalyst to step a) and/or step b),
d) stirring the mixture of steps a) to c) for 2 to 48 h, wherein the carbon donor and the metal oxide precursor, the metal oxide or a combination thereof form a particle,
e) optionally repeating steps a) to c) to form two or more layers of the particle and
f) isolating the formed particles, optionally comprising a pore,
wherein all the steps are performed at a temperature in a range of about −20° C. to about 55° C., preferably in a range of about −5° C. to about 25° C. The saturated solution of the carbon donor and the metal oxide precursor. the metal oxide or the combination thereof, is mixed in step b) for a few minutes up to several hours, in particular in the method for producing a polymer in form of a particle. The mixing takes 1 min to 24 h, 3 min to 20 h, 5 min to 15 h, 10 min to 12 h, 15 min to 10 h, 20 min to 8 h, 30 min to 6 h, 45 min to 5 h, 1 h to 4 h or 2 to 3 h.

The carbon donor of the methods of the present invention is for example selected from the group consisting of a monosaccharide, disaccharide, oligosaccharide, polysaccharide, polyol or a combination thereof.

The metal oxide precursor of the methods of the present invention is for example tetraethyoxysilane (TEOS), tetra-methyl-ortho-silicate (TMOS) and/or a metal oxide selected from the group consisting of an oxide of Si, Ti, Fe, Au, Ag, Al, Cu, Cr, Gd, Zn, Zr, Ru, Rh, Pd, Sn, Cd, Sb, Te, U, Er, Yb, or a combination thereof.

The polycondensation catalyst of the methods of the present invention is for example a basic polycondensation catalyst for example selected from the group consisting of NaOH, KOH, NH₄OH, LiOH, Mg(OH)₂, a basic amino acid, a basic peptide, N,N′-dimethylethylenediamine or a combination thereof. If the amount of the polycondensation catalyst is increased, e.g., by a factor of about 2× to 10× for increasing the number of pores of the particle.

An active agent is for example added to step a) and/or step b) of the methods of the present invention. The active agent is for example in pure form, solid, liquid or gas, dissolved in a hydro-alcoholic solution, dissolved in a water-organic solvent or a combination thereof for incorporating the active agent in the polymer or particle for example in the pore.

Moreover, the present invention is directed to a polymer, gel and particle, respectively, obtainable by a method of the present invention.

The polymer, gel or particle of the present invention comprises a carbon donor such as a carbohydrate, a metal oxide precursor, a metal oxide or a combination thereof, and a polycondensation catalyst, and optionally an active agent, wherein the metal oxide precursor, the metal oxide or the combination thereof forms a scaffold which is covalently connected with carbon of the carbon donor for example wherein 30% to 99% of the scaffold are connected to carbon.

The active agent comprised by the polymer, gel or particle is for example a peptide or protein, enzyme, DNA, RNA, mRNA, siRNA, miRNA, snoRNA, an oligonucleotide, a small molecule or a combination thereof.

The present invention additionally refers to a composition comprising a polymer, gel or particle of the present invention and an excipient such as a pharmaceutically acceptable excipient, a cosmetic acceptable excipient, an agricultural acceptable excipient or a combination thereof.

Furthermore, the present invention relates to a film comprising a polymer, gel, particle or composition of the present invention. The polymer, gel, particle or composition is for example dispersed in the film or located on top of one or both sides of the film.

The polymer, gel, particle, film or composition of the present invention is for example for used as a medicament. Neither the polymer nor the gel, particle, film or composition of the present invention comprises PEG.

The polymer, gel, particle, film or composition of the present invention is for example for use in a method of preventing and/or treating a metabolic disorder/disease such as hyperlipidemia, hypercholesterolemia, hyperglyceridemia, hyperglycemia, insulin resistance, obesity, hepatic steatosis, kidney disease, fatty liver disease, non-alcoholic steatohepatitis, a respiratory disease, a neuronal disease, an inflammatory disease, a viral disease, a cancer disease, a disease of the central nervous system, a cardio-vascular disease or a combination thereof.

Moreover, polymer, gel, particle, film or composition of the present invention is for example for use in a method of treating and/or preventing a diabetes-related complication in a subject. The diabetes-related complication is for example selected from the group consisting of decreased blood flow in the extremities, retinopathy, cardiovascular disorder, peripheral artery disorder, lower limb gangrenous inflammation and a combination thereof. The diabetes is for example selected from the group consisting of Type I diabetes, Type II diabetes, Type II diabetes related to obesity, gestational diabetes and a combination thereof.

The polymer, gel, particle, film or composition of the present invention is for example administered locally or systemically for example orally, sublingually, buccally, intravenously, subcutaneously, intramuscularily, enterally, parenterally, topically, vaginally, topically, rectally, intraocularily or in a combination thereof.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### DESCRIPTION OF THE DRAWINGS

FIG. 1 depicts an example of a reactional scheme for the production of particles according to the present invention. Solution 1 comprises or consists of a saturated carbon donor such as a saccharide-saturated solution of a monosaccharide, oligosaccharide, polysaccharide or a combination thereof. Solution 2 is prepared of a metallic or metal oxide compound or a combination thereof, and/or optionally an organic compound such as a monomer or a polymer, a natural or synthetic drug, an active agent or a combination thereof. Solution 3 comprises or consists of solution of a catalyst such as NaOH, KOH, NH₄OH, etc. in water-alcohol, water-organic solvent or a combination thereof, and optionally a saccharide-saturated solution of a monosaccharide, oligosaccharide, polysaccharide or a combination thereof.

FIG. 2 depicts a TEM image of non-hybrid saccharide particles in a spherical conformation.

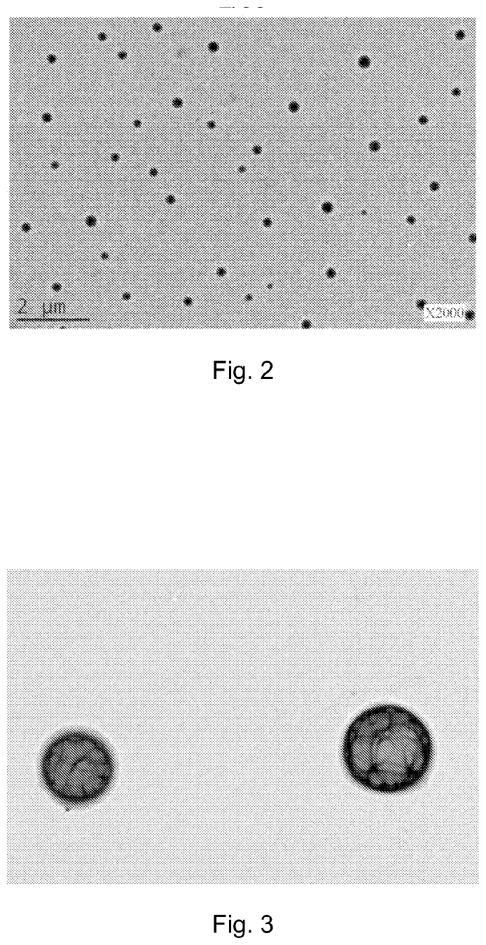

FIG. 3 shows a TEM image of hybrid silica/saccharide particles of the present invention in a spherical conformation.

FIG. 4 shows a SEM image of particles of the present invention in a rice-like conformation.

FIG. 5 shows a SEM image of a gel made out of the biodegradable particles of the present invention.

FIG. 6 shows SEM images of porous particles of the present invention.

FIG. 7A to 7C show TEM images of non-degradable nanoparticles of the prior art incubated with MDCK II (Madin darby canine kidney cells) (FIG. 7A-7C).

FIGS. 8A and 8B show SEM images of insulin-encapsulated sub-microparticles of the present invention (SLIM formulation, AF-type). SLIM is the acronym of: “Sub-Lingual-Insulin-Modality” and AF-type corresponds to a SLIM particle comprising human insulin conceived for a slow release profile. A SLIM-AF sub-microparticle is composed of multiple layers that releases insulin stepwise following the degradation of the particle layer by layer.

FIG. 9 depicts the blood glucose (BG) level in healthy domestic pigs after sublingual application of the SLIM formulation (AF, 5 IU). The blood glucose level declined immediately towards a steady-state level. After stabilization of the BG level for more than 90 min, a glucose-challenge of 3 mL glucose solution (40% w/v) was provided to the animal by subcutaneous administration. This led to an increase of the blood glucose followed by an immediate decline within 10 min towards the previous normalized steady-state level. This suggested a persisting presence of active insulin in the blood of the animal.

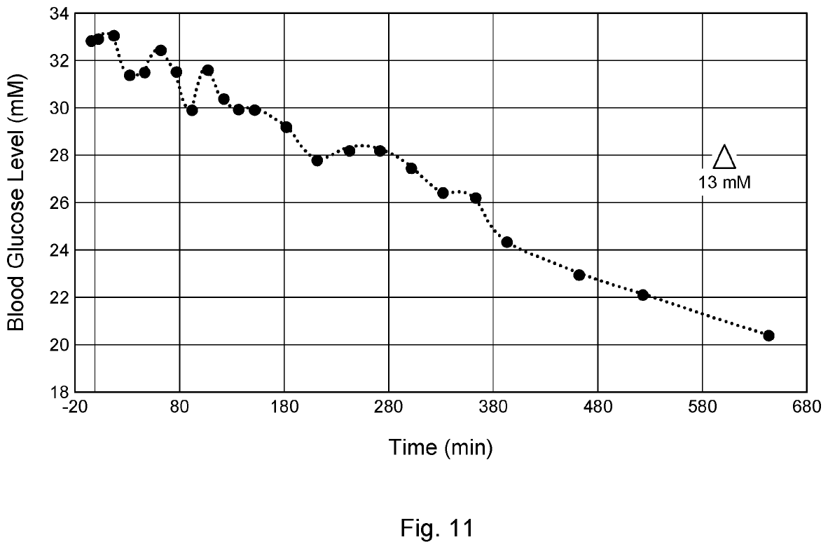

FIG. 10 shows the blood glucose (BG) level in diabetic domestic pigs after sublingual application of the SLIM formulation (BF, 10 IU). BF-type refers to a SLIM particle comprising recombinant human insulin and conceived for a fast release profile. SLIM-BF comprises a nanoporous microparticle loaded with human insulin. The pores of the particle filled with insulin act as a reservoir which can instantly release insulin in a continuous flow once the particles reach the blood stream. The BG quickly dropped by 10 mM within 60 min and subsequently stabilized during approximately an hour.

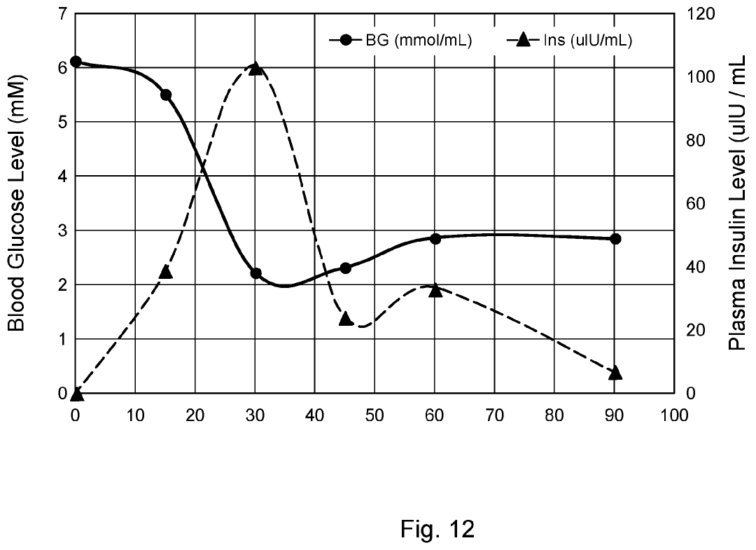

FIG. 11 depicts the blood glucose (BG) level in diabetic domestic pig after sublingual application of the SLIM formulation (BF; 15 IU).

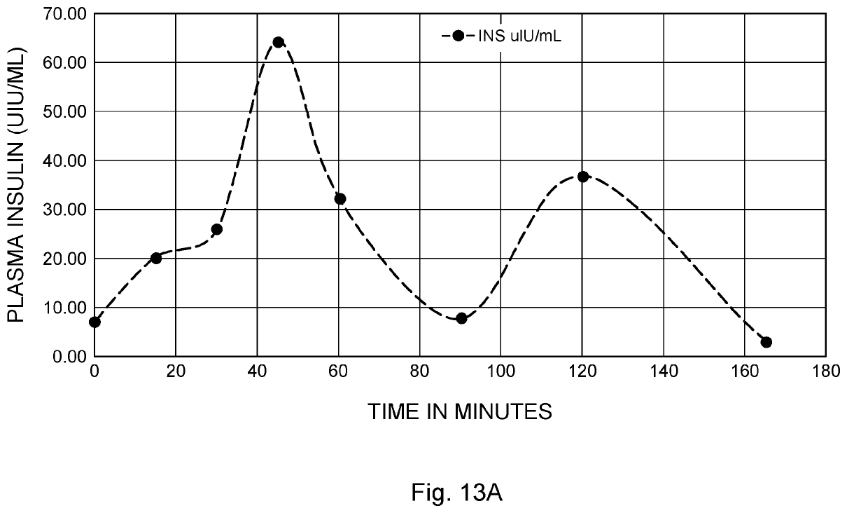

FIG. 12 depicts the blood glucose (BG) level (in mM) and plasma insulin level (in μIU/mL) versus time in minutes after subcutaneous injection of recombinant human insulin (2 IU) in a healthy domestic pig.

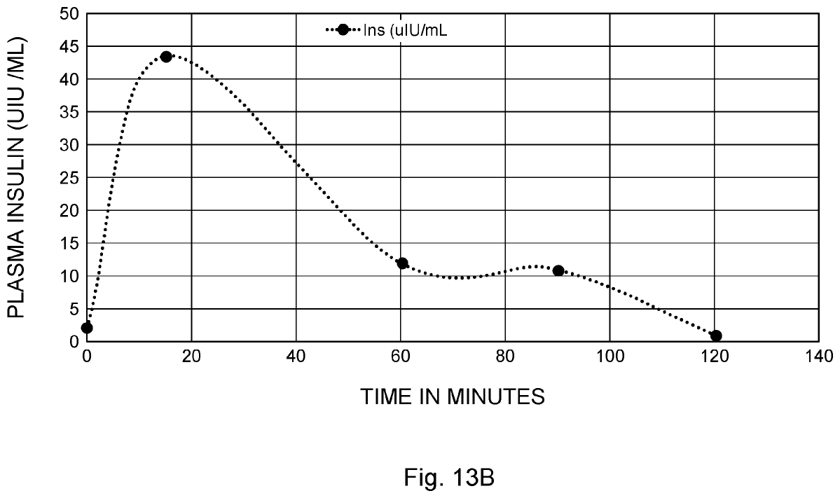

FIGS. 13A and 13B show plasma insulin levels (in μIU/mL) versus time in minutes after sublingual application of SLIM (BF, 3,5 IU) in a diabetic domestic pig showing two maxima of plasma insulin (FIG. 13A) against only one maxima of insulin when commercial insulin (2 IU) was administered subcutaneously to the same diabetic pig (FIG. 13B).

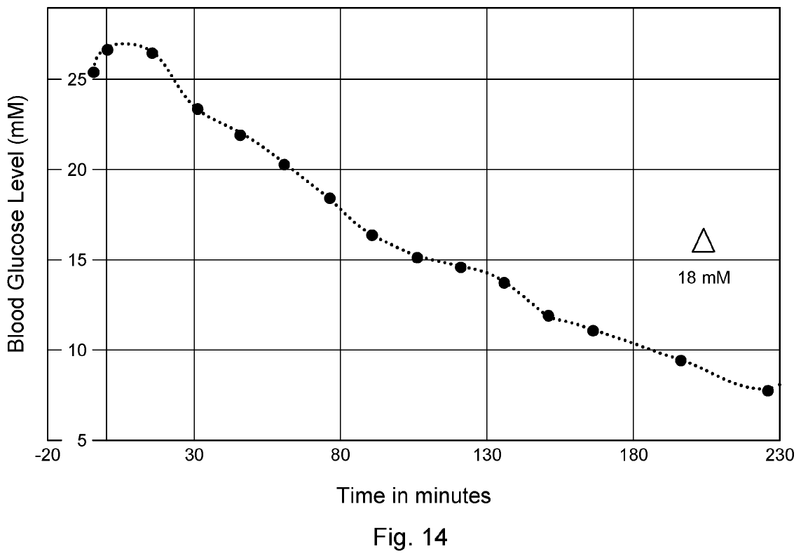

FIG. 14 shows blood glucose (BG) level (in mM) versus time in minutes after sublingual application of a mixture of SLIM (AF-type) and SLIM (BF-type) formulations in a diabetic Göttingen minipig, showing a cumulative effect of the two SLIM formulations bringing the blood glucose to physiological level during about 10 hours after a single dose application.

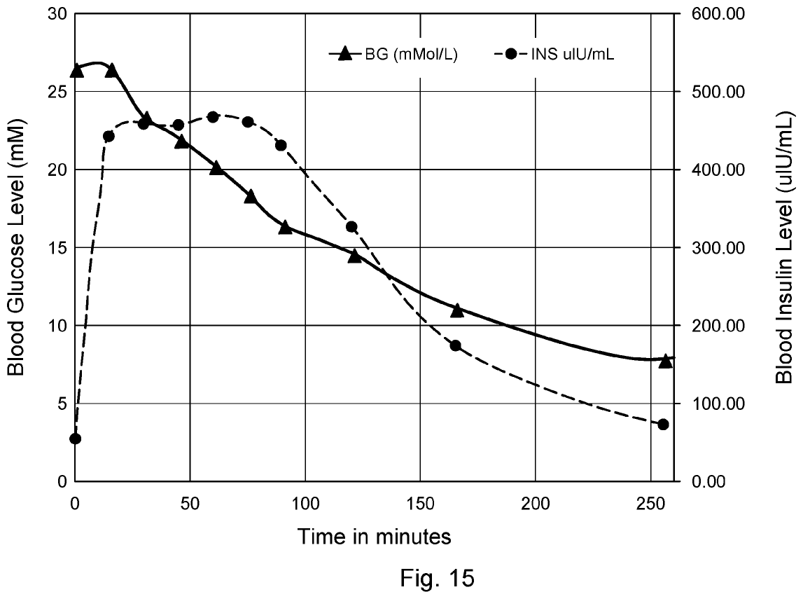

FIG. 15 shows blood glucose (BG) level (in mM) and plasma insulin level (in μIU/mL) versus time in minutes after sublingual application of a mixture of SLIM (AF-type) and SLIM (BF-type) in diabetic Göttingen minipigs, showing the cumulative effect of the two SLIM formulations. The insulin level in plasma reached a maxima level rapidly within 10 min and stabilized at this maximum level for more than an hour.

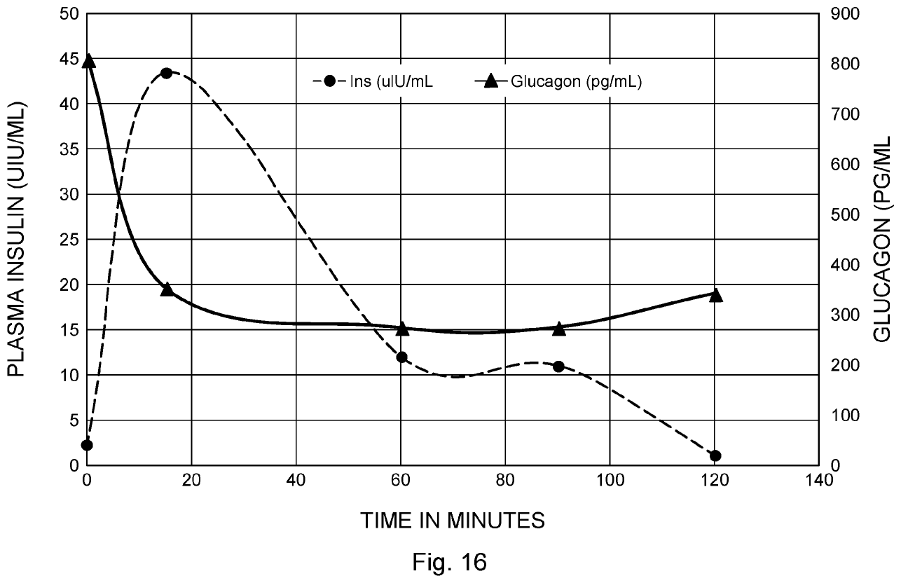

FIG. 16 depicts blood glucagon level (in pg/mL) and plasma insulin level (in μIU/mL) versus time in minutes in a diabetic Göttingen minipig after subcutaneous injection of recombinant human insulin (2 IU) showing only one insulin maximum in plasma.

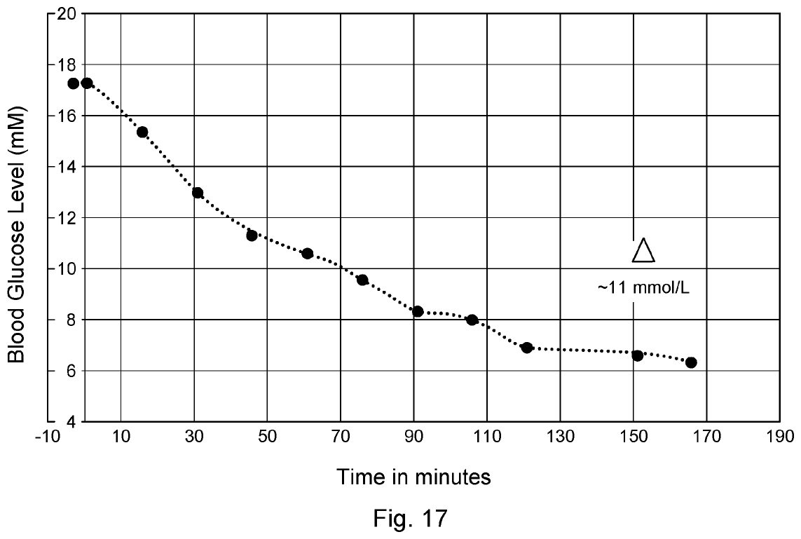

FIG. 17 depicts blood glucose (BG) level (in mM) versus time in minutes in diabetic Göttingen minipigs after subcutaneous injection of recombinant human insulin (10 IU).

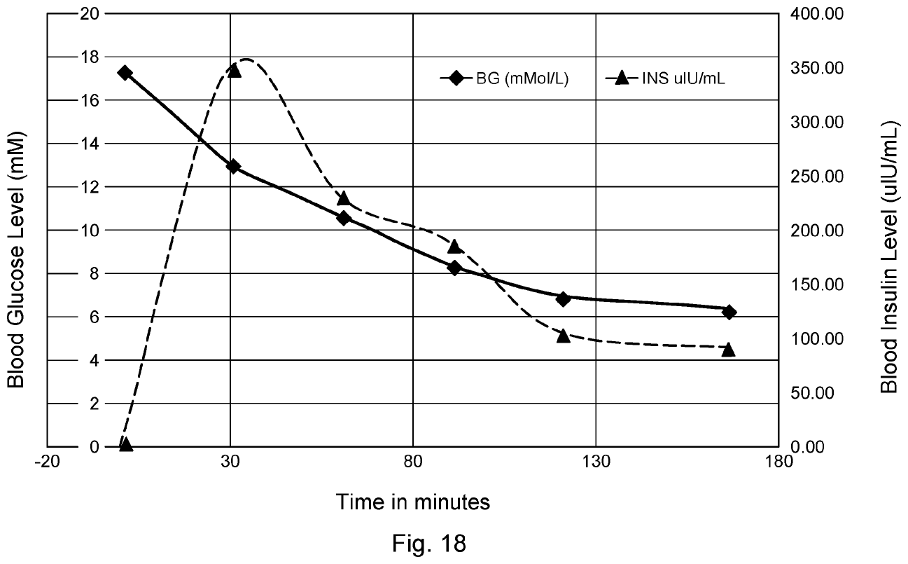

FIG. 18 depicts blood glucose (BG) level (in mM) and plasma insulin level (in μIU/mL) versus time in minutes after subcutaneous injection of recombinant human insulin (10 IU) in a diabetic Göttingen minipig, showing the presence of only one maximum peak of plasma insulin.

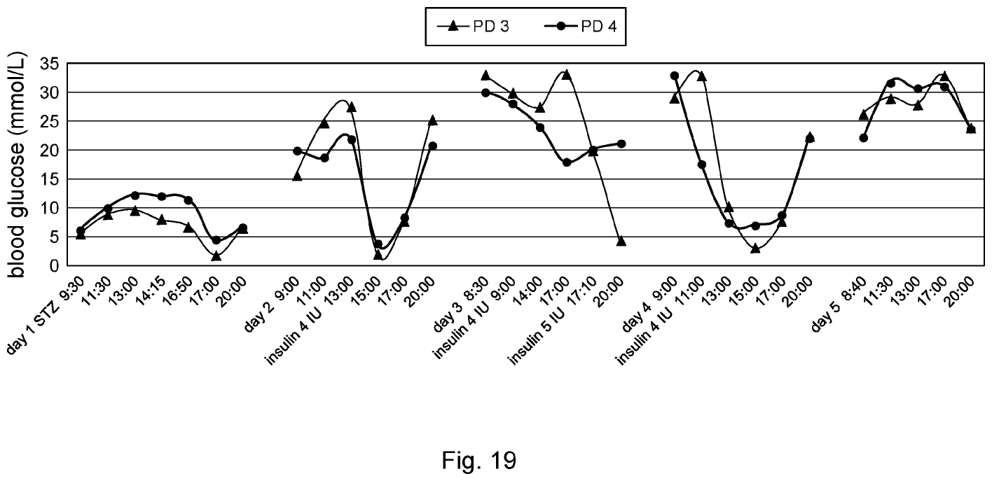

FIG. 19 shows experiments with domestic pigs: during the 5 days follow up of the BG level, a stable STZ diabetic stage has been reached in domestic pigs DP3 and DP4.

FIG. 20 depicts experiments with mini-pigs: during the 4 days follow up of the BG level, a stable STZ diabetic stage has been reached in minipigs P1, P2 and P3.

FIG. 21 depicts the blood glucose (BG) level (in mM) in normoglycemic domestic Pig 3 after sublingual application of the SLIM formulation (AF 2, 2,2 IU) compared to subcutaneous injection of the commercial insulin (Novo Rapid, 2 IU) in normoglycemic domestic Pig 4. AF2 is a SLIM AF-type multilayer particles encapsulating 2.2 IU of recombinant human insulin.

FIG. 22 shows the blood glucose (BG) level (in mM) after sublingual application of the SLIM formulation (AF5, 15 IU) compared to subcutaneous injection of the commercial insulin (Recombinant Human Insulin, 10 IU) in healthy Göttingen minipigs. AF 5 is a SLIM AF-type multilayer particles encapsulating 15 IU of recombinant human insulin.

FIG. 23 depicts the dose effect of human insulin loaded in the SLIM (BF-type) particles in controlling the kinetic of decline of the plasma blood glucose (BG). SLIM (BF-type) particles loaded with 3 different doses of insulin (BF, 5 IU), (BF, 10 IU), (BF, 15 IU), were applied sublingually to the same diabetic domestic Pig 2.

FIG. 24 depicts a dose-response of sublingually administered SLIM-AF-type particles on the plasma blood glucose levels in healthy domestic pigs.

FIG. 25 shows dose-responses of sublingually administered SLIM particles on the plasma blood glucose levels in STZ-diabetic domestic pigs. Pig1 and Pig2 received the same SLIM particle formulation (FX) at a dose of 10 IU and 15 IU, respectively.

FIG. 26 depicts dose-responses of sublingually administered SLIM particles on the plasma blood glucose levels in STZ-diabetic domestic pigs. The same pig (Pig1) received the same SLIM particle formulation (FX) at the same dose of 15 IU on different days.

FIG. 27 depicts a graph showing the large window of efficacy and pharmacokinetics of various SLIM particle formulations of the present invention.

FIGS. 28A and 28B depict dose-response of SLIM particle formulations of the present invention on the plasma blood glucose levels showing a monophasic release profile of insulin in the blood upon sublingual application of the particles. In FIG. 28A 10 IU insulin were administered sublingually in STZ-diabetic domestic pigs, in FIG. 28B 25 IU insulin sublingually in STZ-diabetic domestic pigs.

FIGS. 29A and 29B show serum titles of a spike protein (FIG. 29A) and a mixture of two receptor binding domain (RBD) motives (FIG. 29B) administered to mice via particles of the present invention. The average serum title of the spike protein is 1/20,000 and the average serum title of the RED motives is 1/15,000.

FIG. 30 depicts the antibody serum title of a Spike protein attached to keyhole limpet haemocyanin (KLH) in two different mice which have been immunized with the Spike protein attached to keyhole limpet haemocyanin (KLH) according to the classical immunization technique. The results of the experiment are shown in FIG. 30. The serum title is 1/925.

FIG. 31 depicts the release of insulin from a SLIM (AF-type) particle of the present invention comprising insulin in a concentration of 1.9 IU/mg. The particle comprises for example several layers comprising insulin which is released from the first layer after ca. 16 h, from the second layer after ca. 22 h, and from the third layer after ca. 26 h.

FIG. 32 shows the release of insulin from a SLIM (AF-type) particle of the present invention comprising insulin in a concentration of 0.64 IU/mg. The particle comprises for example several layers comprising insulin which is released from the first layer after ca. 2 h and from the second layer after ca. 3 h.

FIG. 33 depicts the release of insulin from a SLIM (BF-type) particle of the present invention comprising insulin in a concentration of 1.8 IU/mg. The particle comprises for example several layers comprising insulin which is released mostly from the outer layers over a period of 1 h and from the inner layers after ca. 4 h.

### DETAILED DESCRIPTION OF THE INVENTION

The present invention refers to a method for the production of a polymer in form of a gel or particle. The particle is preferably hollow and/or comprises pores. The particle is for example a nanoparticle or a microparticle having a size in the nanomolar or micromolar range, respectively. A particle of the present invention has for example an average particle diameter in the range of about 0.1 nm to about 500 μm, of about 1 nm to about 200 nm or of about 15 nm to about 150 nm (e.g., nanoparticle), of about 200 nm to about 1 μm (e.g., sub-microparticle) or of about 1 μm to about 200 μm (e.g., microparticle). The average particle diameter of the nanoparticles can be modulated by adjusting reaction parameters, particularly temperature, duration and the ratio of inorganic precursor to the carbohydrate and the basic species within the reaction mixture.

As used herein, “average particle diameter” is used to refer to the size of particles in diameter, as measured by conventional particle size analyzers well known to those skilled in the art, such as sedimentation field flow fractionation, photon correlation spectroscopy, laser light scattering or dynamic light scattering technology and by using transmission electron microscope (TEM) or scanning electron microscope (SEM) or X-Ray diffraction (XRD). A convenient automated light scattering technique employs a Horiba LA laser light scattering particle size analyzer or similar device. Such analysis typically presents the volume fraction, normalized for frequency, of discrete sizes of particles including primary particles, aggregates and agglomerates. X-ray diffraction techniques are also widely used which determines the crystal size and conformation and reveals information about the crystallographic structure, chemical composition and physical properties of materials.

The polymer, gel or particle comprises for example an active agent such as a peptide or protein, e.g., a hormone or an enzyme, DNA or RNA and their derivatives such as mRNA, siRNA, miRNA, snoRNA, an oligonucleotide, or a small molecule such as a drug.

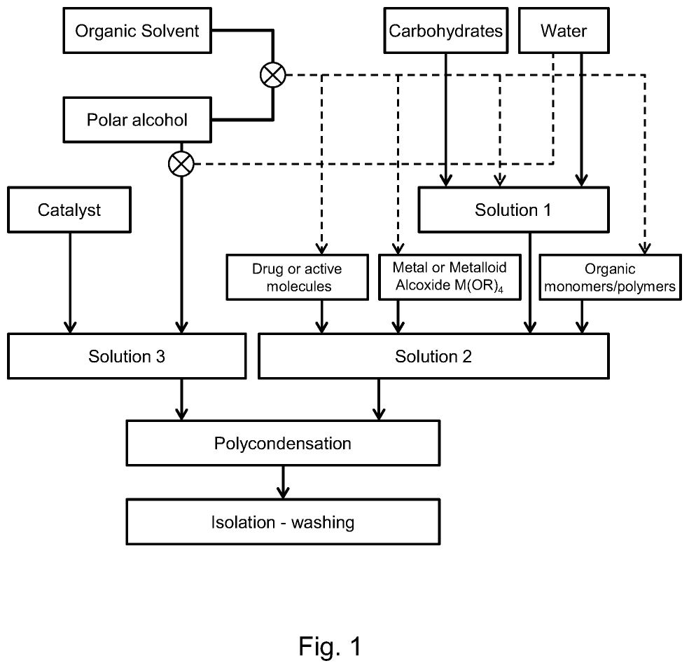

The method of the present invention comprises the steps of preparing a saturated solution of a carbon donor such as a carbohydrate (organic component) which is dissolved in a water/alcohol solvent or a water/alcohol/organic solvent (e.g., FIG. 1). The saturated solution of the carbon donor is mixed with a metal oxide precursor, a metal oxide or a combination thereof (inorganic component). An alcoholic or hydro-alcoholic solution of a polycondensation catalyst is added to the saturated solution of the carbon donor, to the mixture or to both. The method is for example performed at a temperature in the range of about −20° C. to about 65° C., preferably in the range of about −5° C. to about 25° C. Stirring of the mixture results in the formation of a particle; if the mixture is not stirred and optionally the amount of solvent is reduced, a gel is formed.

It is to be understood that any modification in the type, the manner, and the order of addition of the components in the steps of the method for preparing the polymer, gel or particle which is obvious to the person skilled in the art is also inclusive to the present invention.

Optionally an active agent is added to the saturated solution of a carbon donor, to the mixture or both. The active agent, for example interacting with the organic and inorganic component of the polymer, gel or particle, has a state of reduced activity. Due to this state of the active agent a high concentration of the active agent can be received by the polymer, gel or particle. Further, the state of reduced activity of the active agent leads to reduced degradation, deactivation or complexation of the active agent during the retention time in the polymer, gel or particle.

The retention time of the active agent in the polymer, gel or particle and the stability of the polymer, gel or particle, respectively, depends on the ratio of organic:inorganic component. The higher the ratio of the organic component, the faster the degradation of the polymer, gel or particle and the faster the release of the active agent, respectively. The higher the ratio of the inorganic compound the higher the stability of the polymer, gel or particle and the slower the release of the active agent, respectively.

Incorporation of the active agent in the polymer, gel or particle can be done by any suitable method. Eventually, the pH of the saturated solution of the carbon donor such as one or more saccharide(s) or oligosaccharide(s) and/or the pH of the polycondensation reaction medium and/or of the additive is adjusted according to the active agent to be incorporated for example encapsulated in the particle.

The incorporation of the active molecule into a composition further comprises a constituent element, which may be a preservative, a stabilizer, an adjuvant, a light sensitizer,an energizer, an additive that protects against the degradation of biologically active molecules, e.g., a saturated solution of saccharide(s) or oligosaccharide(s). This has the advantage of preserving the stability and biological activity of the biomolecules during encapsulation.

The present invention is further directed to the polymer, gel or particle obtainable by the method of the present invention. In addition, it relates to a composition and a film, respectively, comprising a polymer, gel or particle of the present invention. The polymer, gel or particle and/or composition is for example dispersed in the film or located on top of one or both sides of the film.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention. The term “about” means a variation of up to plus and/or minus 10% of the specific value.

In the following the present invention is discussed in more detail and embodiments of the invention are listed. It should be understood that the elements of the embodiments may be combined in any manner and in any number to create additional embodiments. The variously described examples and embodiments should not be construed to limit the present invention to only the explicitly described embodiments. Furthermore, any permutations and combinations of all described embodiment in this application should be considered disclosed by the description of the present application unless the context indicates otherwise. Embodiments of the invention are for example:

1. Method for the production of a polymer such as a gel comprising the steps:
a) preparing a saturated solution of a carbon donor such as a carbohydrate, the carbon donor is dissolved in a water/alcohol solvent or water/alcohol/organic solvent wherein the ratio of water:alcohol is from 20:80 to 80:20 or wherein the ratio of water:alcohol:organic solvent is from 5:80:15 to 80:15:5,
b) mixing the saturated solution of step a) with a metal oxide precursor, a metal oxide or a combination thereof,
c) adding an alcoholic or hydro-alcoholic solution of a polycondensation catalyst to step a) and/or step b),
wherein all the steps are performed at a temperature in a range of about −20° C. to about 65° C., preferably about −5° C. to about 25° C. and the carbon donor and the metal oxide precursor, the metal oxide or a combination thereof form a gel,
d) optionally additives are added in step a), b) or c). The additive is for example a pH-responsive polymer, e.g., a polycarboxylic acid such as PAA (polyacrylic acid), PMA (polymethacrylic acid), or polysufonamides, a cationic polyelectrolyte such as PLL (poly L-Lysine), PEI (poly(ethylenimine), or chitosan or a combination thereof.

2. Method for the production of a polymer such as a particle comprising the steps:
a) preparing a saturated solution of a carbon donor such as a carbohydrate, the carbon donor is dissolved in a water/alcohol solvent or water/alcohol/organic solvent wherein the ratio of water:alcohol is from 20:80 to 80:20 or wherein the ratio of water:alcohol:organic solvent is from 5:80:15 to 80:15:5,
b) mixing the saturated solution of step a) with a metal oxide precursor, a metal oxide or a combination thereof,
c) adding an alcoholic or hydro-alcoholic solution of a polycondensation catalyst to step a) and/or step b),
d) stirring the mixture of steps a) to c) for 2 to 48 h, wherein the carbon donor and the metal oxide precursor, the metal oxide or a combination thereof form a particle,
e) optionally repeating steps a) to c) to form two or more layers of the particle and
f) isolating the formed particles, optionally comprising a pore,
wherein all the steps are performed at a temperature in a range of about −20° C. to about 65° C., preferably in a range of about −5° C. to about 25° C.

3. Method according to embodiment 1 or 2, wherein the carbon donor is selected from the group consisting of a monosaccharide, disaccharide, oligosaccharide, polysaccharide, polyol or a combination thereof.

4. Method according to any one of embodiments 1 to 3, wherein the monosaccharide is glucose, fructose, galactose or a combination thereof.

5. Method according to any one of embodiments 1 to 4, wherein the disaccharide is maltose, sucrose, lactose, or a combination thereof.

6. Method according to any one of embodiments 1 to 5, wherein the disaccharide is maltose.

7. Method according to any one of embodiments 1 to 5, wherein the disaccharide is lactose.

8. Method according to any one of embodiments 1 to 7, wherein the oligosaccharide is glycan, fructooligosacchride, galactooligosaccharide, lactulose, raffinose or a combination thereof.

9. Method according to any one of embodiments 1 to 8, wherein the polysaccharide is starch, dextrin, chitin, cellulose or a combination thereof.

10. Method according to any one of embodiments 1 to 9, wherein the metal oxide precursor is tetraethyoxysilane (TEOS), tetra-methyl-ortho-silicate (TMOS), and/or a metal oxide selected from the group consisting of an oxide of Si, Ti, Fe, Au, Ag, Al, Cu, Cr, Gd, Zn, Zr, Ru, Rh, Pd, Sn, Cd, Sb, Te, U, Er, Yb, or a combination thereof.

11. Method according to any one of embodiments 1 to 10, wherein the metal oxide is SiO₂, TiO₂, FeO, Fe₂O₃, Au₂O₃, Ag₂O, Ag₂O₃, Ag₄O₄, Al₂O₃, CuO, Cu₂O, CrO, Cr₂O₃, CrO₂, Gd₂O₃, ZnO, ZrO₂, RuO₂, RhO₂, Rh₂O₃, PdO, SnO, SnO₂, CdO, Sb₂O₃, TeO₂, TeO₃, UO₂, U₂O₅, U₃O₈, Er₂O₃, Yb₂O₃ or a combination thereof.

12. Method according to any one of embodiments 1 to 11, wherein the metal oxide is SiO₂.

13. Method according to any one of embodiments 1 to 12, wherein polycondensation catalyst is a basic polycondensation catalyst for example selected from the group consisting of NaOH, KOH, NH₄OH, LiOH, Mg(OH)₂, a basic amino acid, a basic peptide, N,N′-dimethylethylenediamine or a combination thereof.

14. Method according to any one of embodiments 1 to 13, wherein polycondensation catalyst is NaOH, KOH, NH₄OH or a combination thereof.

15. Method according to any one of embodiments 1 to 14, wherein the amount of the polycondensation catalyst is increased by a factor of about 2× to 10× for increasing the number of pores of the gel.

16. Method according to any one of embodiments 1 to 14, wherein the amount of the polycondensation catalyst is increased by a factor of about 2× to 10× for increasing the number of pores of the particle.

17. Method according to any one of embodiments 1 to 16, wherein an active agent is added to step a) and/or step b).

18. Method according to any one of embodiments 1 to 17, wherein the active agent is in pure form, solid, liquid or gas.

19. Method according to any one of embodiments 1 to 18, wherein the active agent is dissolved in a hydro-alcoholic solution, dissolved in a water-organic solvent or a combination thereof for incorporating the active agent in the polymer, gel or particle for example in the pore.

20. Method according to any one of embodiments 1 to 19, wherein the alcohol is selected from the group consisting of methanol, ethanol, propanol or a combination thereof.

21. Method according to any one of embodiments 1 to 20, wherein the alcohol is pure or anhydrous methanol, ethanol, propanol or a combination thereof.

22. Method according to any one of embodiments 1 to 21, wherein the alcohol comprises ammonium hydroxide.

23. Method according to embodiment 22, wherein the ammonium hydroxide is an about 20% to 30% aqueous solution of ammonium hydroxide.

24. Method according to embodiment 22 or 23, wherein the alcohol comprises from about 1% v/v to about 10% v/v of the aqueous ammonium hydroxide solution, from about 5% v/v to about 7% v/v or 6.66% v/v.

25. Method according to any one of embodiments 1 to 24, wherein the saturated solution is filtered to eliminate insoluble particulates.

26. Method according to any one of embodiments 2 to 25, wherein the particle is isolated via centrifugation.

27. Method according to any one of embodiments 2 to 26, wherein the isolated particle is washed to remove unreacted carbohydrate such as a monosaccharide, disaccharide, oligosaccharide, polysaccharide or a combination thereof

28. Method according to any one of embodiments 2 to 27, wherein the isolated particle is washed to remove unreacted metal oxide precursor, metal oxide or a combination thereof.

29. Method according to any one of embodiments 2 to 28, wherein the isolated particle is washed with an organic solvent.

30. Method according to any one of embodiments 27 to 29, wherein the organic solvent is an aromatic compound, an alcohol, an ester, an ether, a ketone, an amine, a nitrated hydrocarbon or a halogenated hydrocarbon.

31. Method according to embodiment 29 or 30, wherein the organic solvent is selected from the group consisting of benzene, toluene, ethanol, methanol, butanol, propanol, pentane, hexane, heptane, acetone, acetic acid, chloroform, cyclohexane, pyridine, tetrahydrofuran, xylene or a combination thereof.

32. Method according to any one of embodiments 1 to 31, wherein the active agent is a peptide or protein, enzyme, DNA, RNA, mRNA, siRNA, miRNA, snoRNA, an oligonucleotide, a small molecule or a combination thereof.

33. Method according to any one of embodiments 1 to 32, wherein the active agent is a hormone such as insulin.

34. Method according to any one of embodiments 1 to 32, wherein the active agent is an incretin, a glucagon-like peptide (GLP-1) agonist and/or its analogue.

35. Method according to any one of embodiments 1 to 32, wherein the active agent is a microorganism or a fragment of a microorganism such as a virus, virus fragment, bacterium, bacterium fragment or a combination thereof.

36. Method according to embodiment 35, wherein the virus fragment is a spike mRNA, a spike protein or a part thereof.

37. Method according to embodiment 35, wherein the virus fragment is a cell receptor or a part thereof.

38. Method according to embodiment 37, wherein the cell receptor or a part thereof comprises a binding motif for the microorganism such as a virus or bacterium.

39. Method according to embodiment 38, wherein the binding motive comprises or consists of GNYNYLYRLFRKSNLKPFERDISTEIYQAGSTPC (SEQ ID NO.1; e.g., 34aa, 4 kDa (part N-Nter)), CYFPLQSYGFQPTNGVGYQPYR (SEQ ID NO.2; 22aa, 2.6 kDa (part C-Nter)) or a combination thereof.

40. Method according to any one of embodiments 1 to 39, wherein the active agent is loaded in the pores of the polymer or particle.

41. Method according to embodiment 40, wherein the active agent loaded in the pore is selected from the group of a natural or synthetic molecule, an enzyme, a protein, a peptide, an antibody, an oligonucleotide, a gene, a gene fragment, an antigen, a vaccine, a cellular organism such as micro-organisms, such as bacteria, yeasts, fungi, algae, cells of animal or plant origin or a combination thereof.

42. Polymer obtainable by a method according to any one of embodiments 1 to 41 or particle obtainable by a method according to any one of embodiments 2 to 41.

43. Polymer or particle according to embodiment 42 comprising a carbon donor such as a carbohydrate, a metal oxide precursor, a metal oxide or a combination thereof, and a polycondensation catalyst, and optionally an active agent.

44. Polymer or particle according to embodiment 42 or 43, wherein the metal oxide precursor, the metal oxide or the combination thereof forms a scaffold which is covalently connected with carbon of the carbon donor for example wherein 30% to 99% of the scaffold are connected to carbon of the carbon donor.

45. Polymer or particle according to any one of embodiments 42 to 44, wherein the carbon donor is selected from the group consisting of a monosaccharide, disaccharide, oligosaccharide, polysaccharide, polyol or a combination thereof.

46. Polymer or particle according to any one of embodiments 42 to 45, wherein the monosaccharide is glucose, fructose, galactose or a combination thereof.

47. Polymer or particle according to any one of embodiments 42 to 46, wherein the disaccharide is maltose, sucrose, lactose, or a combination thereof.

48. Polymer or particle according to any one of embodiments 42 to 47, wherein the disaccharide is maltose.

49. Polymer or particle according to any one of embodiments 42 to 47, wherein the oligosaccharide is glycan, fructooligosacchride, galactooligosaccharide, lactulose, raffinose or a combination thereof.

50. Polymer or particle according to any one of embodiments 43 to 50, wherein the polysaccharide is starch, dextrin, chitin, cellulose or a combination thereof.

51. Polymer or particle according to any one of embodiments 42 to 50, wherein the metal oxide precursor is tetraethyoxysilane (TEOS), tetra-methyl-ortho-silicate (TMOS), and/or a metal oxide selected from the group consisting of an oxide of Si, Ti, Fe, Au, Ag, Al, Cu, Cr, Gd, Zn, Zr, Ru, Rh, Pd, Sn, Cd, Sb, Te, U, Er, Yb, or a combination thereof.

52. Polymer or particle according to any one of embodiments 42 to 51, wherein the metal oxide is SiO₂, TiO₂, FeO, Fe₂O₃, Au₂O₃, Ag₂O, Ag₂O₃, Ag₄O₄, Al₂O₃, CuO, Cu₂O, CrO, Cr₂O₃, CrO₂, Gd₂O₃, ZnO, ZrO₂, RuO₂, RhO₂, Rh₂O₃, PdO, SnO, SnO₂, CdO, Sb₂O₃, TeO₂, TeO₃, UO₂, U₂O₅, U₃O₈, Er₂O₃, Yb₂O₃ or a combination thereof.

53. Polymer or particle according to any one of embodiments 42 to 52, wherein the metal oxide is SiO₂.

54. Polymer or particle according to any one of embodiments 42 to 53, wherein polycondensation catalyst is a basic polycondensation catalyst for example selected from the group consisting of NaOH, KOH, NH₄OH, LiOH, Mg(OH)₂, a basic amino acid, a basic peptide, N,N′-dimethylethylenediamine or a combination thereof.

55. Polymer or particle according to any one of embodiments 42 to 54, wherein polycondensation catalyst is NaOH, KOH, NH₄OH or a combination thereof.

56. Polymer or particle according to any one of embodiments 42 to 55, wherein the polymer or the particle is characterized by controlled release selected from the group consisting of mono-, biphasic or multiphase release profile.

57. Polymer or particle according to any one of embodiments 42 to 56, wherein the active agent is a peptide or protein, enzyme, DNA, RNA, mRNA, siRNA, miRNA, snoRNA, an oligonucleotide, a small molecule or a combination thereof.

58. Polymer or particle according to any one of embodiments 42 to 57, wherein the active agent is a hormone such as insulin.

59. Polymer or particle according to any one of embodiments 42 to 57, wherein the active agent is an incretin, a glucagon-like peptide (GLP-1) agonist and/or its analogue.

60. Polymer or particle according to any one of embodiments 42 to 57, wherein the active agent is a microorganism or a fragment of a microorganism such as a virus, virus fragment, bacterium, bacterium fragment or a combination thereof.

61. Polymer or particle according to embodiment 60, wherein the Virus fragment is a spike mRNA, a spike protein or a part thereof.

62. Polymer or particle according to embodiment 60, wherein the Virus fragment is a cell receptor or a part thereof.

63. Polymer or particle according to embodiment 62, wherein the cell receptor or a part thereof comprises a binding motif for the microorganism such as a Virus or bacterium.

64. Polymer or particle according to embodiment 63, wherein the binding motive comprises or consists of GNYNYLYRLFRKSNLKPFERDISTEIYQAGSTPC (SEQ ID NO.1; e.g., 34aa, 4 kDa (part N-Nter)), CYFPLQSYGFQPTNGVGYQPYR (SEQ ID NO.2; 22aa, 2.6 kDa (part C-Nter)) or a combination thereof.

65. Polymer or particle according to any one of embodiments 42 to 64, wherein the active agent is loaded in the pores of the polymer or particle.

66. Composition comprising a polymer or particle according to any one of embodiments 42 to 65 and an excipient such as a pharmaceutically acceptable excipient, a cosmetic acceptable excipient, an agricultural acceptable excipient or a combination thereof.

67. Composition according to embodiment 66, wherein the composition is a powder, capsule, microcapsules, tablet, liquid, suspension, lotion, paste, spray, foam, roll-on, oil, cream, gel, ointment, film, sheet, patch, deodorant, an aerosol, or a combination thereof.

68. Film comprising a polymer or particle according to any one of embodiments 42 to 65 or a composition according to embodiment 66 or 67.

69. Film according to embodiment 68, wherein the polymer, particle and/or composition is dispersed in the film or located on top of one or both sides of the film.

70. Polymer or particle according to any one of embodiments 42 to 65 for use as a medicament.

71. Composition according to embodiment 66 or 67 for use as a medicament.

72. Film according to embodiment 68 or 69 for use as a medicament.

73. Polymer or particle according to any one of embodiments 42 to 65 and 70, composition according to any one of embodiments 66, 67 or 71, or film according to any one of embodiments 68, 69 or 71 for use in a method of preventing and/or treating a metabolic disorder/disease such as hyperlipidemia, hypercholesterolemia, hyperglyceridemia, hyperglycemia, insulin resistance, obesity, hepatic steatosis, kidney disease, fatty liver disease, non-alcoholic steatohepatitis, a respiratory disease, an inflammatory disease, obesity, a viral disease, a cancer disease, a disease of the central nervous system, a cardio-vascular disease or a combination thereof.

74. Polymer, particle, composition or film for use according to embodiment 73, wherein the metabolic disease is diabetes.

75. Polymer, particle, composition or film for use according to embodiment 73, wherein the cancer disease is brain cancer, breast cancer, melanoma or colon cancer.

76. Polymer, particle, composition or film for use according to embodiment 73, wherein the viral disease is Covid such as Covid-19.

77. Polymer, particle, composition or film according to any one of the previous embodiments, wherein the polymer, particle, composition or film is administered locally or systemically.

78. Polymer, particle, composition or film according to embodiment 77, wherein the polymer, particle, composition or film is administered orally, sublingually, buccally, intravenously, subcutaneously, intramuscularily, enterally, parenterally, topically, vaginally, rectally, intraocularily or a combination thereof.

79. Method according to any one of embodiments 1 to 41, wherein the mixing in step b) takes a few minutes to several hours.

80. Method according to any one of embodiments 1 to 41, wherein the mixing in step b) takes 1 min to 24 h, 3 min to 20 h, 5 min to 15 h, 10 min to 12 h, 15 min to 10 h, 20 min to 8 h, 30 min to 6 h, 45 min to 5 h, 1 h to 4 h or 2 to 3 h.

The polymer, gel or particle of the present invention is biosoluble and therefore not biopersistent. It degrades over time in a biological system e.g. cell system, biological fluid (or its simulants), in an animal, or in a body of a subject, wherein the organic component fully dissolves in the biological environment and the inorganic component is degraded into significantly small entities of less than 5 nm that are physiologically resorbed and/or quickly released from the body of the subject.

The biosoluble polymer, gel or particle of the present invention is for example degraded enzymatically. The covalent bonds between the organic and the inorganic components are broken enzymatically. Enzymes breaking these bonds are for example from, but not limited to, the group of cathepsin B, cathepsin C, and cathepsin D, glycosidases, lysozyme, hydrolases acids, acyl-CoA dehydrogenase, acetyl-CoA C-acyltransferase, hexokinase, aldolase, enolase, pyruvate kinase, pyruvate dehydrogenase, citrate synthase, aconitase, isocitrate dehydrogenase, succinyl-CoA synthetase, succinate dehydrogenase, fumarase, malate dehydrogenase, NADH dehydrogenase (complex I), succinate dehydrogenase (complex II), coenzyme Q-cytochrome c reductase (complex III), cytochrome c oxidase (complex IV), translocase ATP/ADP, ATP synthase or a combination thereof.

The particle comprises or consists of one or more layers for example 1 to 10 layers, 2 to 8 layers, or 3 to 5 layers. The ratio of organic:inorganic components are identical in each layer or differ in some or all layers. Each layer comprises for example an active agent or some layers comprise an active agent. The active agent is the same in each layer or different in some or all layers. Each layer comprises the active agent for example in the same or different concentration. The active agent is for example loaded in a layer in an amount of about 0.1% w/w to about 99% w/w. The particle of the present invention provides for example a combination of fast and slow release of the active agent. Layered particles result for example in a continuous, extended effect of the active agent since the active agent is released via a step-by-step degradation of each layer after layer of the particle.

The organic component is the carbon donor. It is for example a carbohydrate such as a monosaccharide (e.g., glucose, fructose, galactose), disaccharide (e.g., maltose, sucrose, lactose), oligosaccharide (comprising 3 to about 10 monosaccharides, e.g., glycan, fructooligosacchride, galactooligosaccharide, lactulose, raffinose), polysaccharide (e.g., starch, dextrin, chitin, cellulose), polyol or a combination thereof. Each of the saccharides is for example a natural saccharide, a synthetic saccharide or a semi-synthetic saccharide. The term saccharide includes monosaccharide, disaccharide, oligosaccharide and polysaccharide.

The polysaccharide is selected from the group consisting of starch, dextrin, cellulose, chitin, a branched alpha glucan, a branched beta glucan and derivatives thereof.

The saccharide such as an oligosaccharide or a polysaccharide is for example a naturally-occurring saccharide, a naturally-occurring branched saccharide, a synthetic saccharide or a synthetic branched saccharide.

The saccharide is for example selected from the group consisting of glucose, fructose, sucrose, maltose, galactose, trehalulose, lactose, mannose, isomaltulose, mannitol, sorbitol, lactose, amylose, starch, starch derivatives, pectin, amylopectin, glycogen, cyclodextrin, cellulose, naturel and synthetic edulcorates, aspartame, sucralose, saccharine, agave syrup, stevia, honey, edible syrup, or a combination thereof.

The polysaccharide refers for example to a polymer formed from about 500 monomers linked to each other by hemiacetal or glycosidic bonds and may contain as many as 100,000 monomers or more. The polysaccharide is for example either a straight chain, singly branched, or multiply branched wherein each branch may have additional secondary branches. The monomer is for example a standard D- or L-cyclic sugar in the pyranose (6-membered ring) or furanose (5-membered ring) form such as D-fructose and D-galactose, respectively, or a cyclic sugar derivative, for example an amino sugar such as D-glucosamine, deoxy sugar such as D-fucose or L-rhamnose, sugar phosphate such as D-ribose-5-phosphate, sugar acid such as D-galacturonic acid, or a multi-derivatized sugar such as N-acetyl-D-glucosamine, N-acetylneuraminic acid (sialic acid), or N-sulfato-D-glucosamine. Polysaccharide preparations comprise for example molecules that are heterogeneous in molecular weight. Polysaccharides include for example galactomannans and galactomannan derivatives; galacto-rhamnogalacturons and galacto-rhamnogalacturon derivatives, and galacto-arabinogalacturon and galacto-arabinogalacturon derivatives.

The inorganic component is the metal oxide precursor (e.g., tetraethyoxysilane (TEOS) or tetra-methyl-ortho-silicate (TMOS)), a metal oxide (e.g., of Si, Ti, Fe, Au, Ag, Al, Cu, Cr, Gd, Zn, Zr, Ru, Rh, Pd, Sn, Cd, Sb, Te, U, Er, Yb) or a combination thereof. In more detail, metal oxides are for example SiO₂, TiO₂, FeO, Fe₂O₃, Au₂O₃, Ag₂O, Ag₂O₃, Ag₄O₄, Al₂O₃, CuO, Cu₂O, CrO, Cr₂O₃, CrO₂, Gd₂O₃, ZnO, ZrO₂, RuO₂, RhO₂, Rh₂O₃, PdO, SnO, SnO₂, CdO, Sb₂O₃, TeO₂, TeO₃, UO₂, U₂O₅, U₃O₈, Er₂O₃, Yb₂O₃ or a combination thereof.

The inorganic component of the present invention is for example selected from the group consisting of silica, alkaline metals, alkaline earth metals, transition metals, especially zinc, calcium, magnesium, titanium, silver, aluminum, or lanthanides, their salts, hydrates, as well as combinations thereof. The inorganic material is for example in the form of metal, metalloid, metal oxide, alkoxide, oxide, acetate, oxalate, urate, or nitrate.

The ratio of the organic:inorganic component is for example a range from about 0.001% to about 99.99% and from about 99.99% to about 0.001%, respectively, more preferably from about 35% to about 65%.

Any type of active agent is packable in the particle of the present invention for example in the hollow inside of the particle, in the pores of the particle walls or in in both. The active agent is for example loaded in the particle in an amount of about 0.01% w/w to about 99,9% w/w, about 0.1% w/w to about 95% w/w, about 1% w/w to about 90% w/w, about 10% w/w to about 85% w/w, about 20% w/w to about 80% w/w, about 30% w/w to about 75% w/w, about 40% w/w to about 70% w/w, about 50% w/w to about 60% w/w.

The active agent is preferably mixed with the water/alcohol mixture, water/organic solvent mixture or a combination thereof between −20° C. to 65° C., −5° C. to 25° C., 1° C. to 10° C., 0° C. to 5° C., 1° C. or 4 ° C. The frozen conformation of the active agent simplifies the loading of the polymer, gel or particle with the active agent. In addition, a higher amount of active agent is loadable in the polymer, gel or particle. Once the active agent in the frozen conformation is loaded in the polymer, gel or particle, the polymer, gel or particle is storable at room temperature and the biological activity of the loaded active agent is preserved for longer storage without the need for laborious and expensive cooling logistics.

The water/alcohol mixture or the water/organic solvent mixture of the active agent comprises for example the water and alcohol or the water and organic solvent in a ratio from about 0.001% v/v to about 99% v/v, preferably in a ratio from about 20% v/v to about 80% v/v.

Examples for an active agent are selected from the group consisting of, but not limited to antibiotics, antiviral agents, anti-fungals, analgesics, anorexics, antipsoriatics and acne treatment agents, anti-herpes agents, antihelminthic, antiarthritics, antiasthmatic agents, anticonvulsants, antidepressants, antidiabetic agents, antidiarrheals, antihistamines, anti-inflammatory agents, antimigraine preparations, antinauseants, antiandrogens, antisyphilictic agents, antineoplastics, antiparkinsonism drugs, antipruritic, antipsychotics, antipyretics, antispasmodics, anticholinergics, sympathomimetics, xanthine derivatives, cardiovascular preparations including potassium and calcium channel blockers, beta-blockers, alpha-blockers, and antiarrhythmics, antihypertensives, diuretics and antidiuretics, vasodilators including general coronary, peripheral and cerebral, central nervous system stimulants, vasoconstrictors, cough and cold preparations, including decongestants, hormones such as testosterone, estradiol and other steroids, including corticosteroids, hypnotics, immunosuppressives, muscle relaxants, parasympatholytics, psychostimulants, dermatitis herpetoformis suppressants, topical protectants, mosquito repellants, anti-lice agents, sedatives, tranquilizers, macromolecules such as proteins, enzymes, polypeptides, polysaccharides, vaccines, antigens, antibodies, amino acids, nucleic acids such as RNA (e.g., siRNA, mRNA), DNA, oligonucleotides, a fatty acid, metal ions, a chelating agent, a light active agent such as a fluorescent or a luminescent compound, a natural drug, a synthetic drug, a cosmetic compound or an agricultural compound and combinations thereof. An agricultural compound is for example nutrient, pesticide, insecticide, fertilizer, fungicide, biostimulant, insect repellant, fumigant, nematode repellent or a combination thereof.

An active agent such as an antiviral agent is for example selected from group of but not limited to acyclovir, ganciclovir, famciclovir, foscamet, inosine-(dimepranol-4-acetamidobenzoate), valganciclovir, valacyclovir, cidofovir, brivudin, antiretroviral active ingredients (nucleoside analog reverse-transcriptase inhinbitors and derivatives) such as lamivudine, zalcitabine, didanosine, zidovudin, tenofovir, stavudin, abacavir, non-nucleoside analog reverse-transcriptase inhibitors such as amprenavir, indinavir, saquinavir, lopinavir, ritonavir, nelfinavir, amantadine, ribavirin, zanamivir, oseltamivir as well as any combinations thereof.

An active agent such as an antifugal agent is for example selected from but not limited to allyamines (amrolfine, butenafine, naftifine, terbinafine), azoles (ketoconazole, fluconazole, elubiol, econazole, econaxole, itraconazole, isoconazole, imidazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, flutrimazole, polyenes (nystatin, pimaricin, amphotericin B), pyrimidines (flucytosine), tetraenes (natamycin), thiocarbamates (tolnaftate), sulphonamides (mafenide, dapsone), glucan synthesis inhibitors (caspofungin), benzoic acid compounds, complexes and derivatives thereof (actofunicone) and other systemic or mucosal (griseofluvin, potassium iodide, gentian violet) and topical drugs (ciclopirox, ciclopirox olamine, haloprogin, undecylenate, silver sulfadiazine, undecylenic acid, undecylenic alkanolamide, Carbol-Fuchsin) as well as any combinations thereof.

An active agent such as an antibacterial agent is for example selected from but not limited to aclacinomycin, actinomycin, anthramycin, azaserine, azithromycin, bleomycin, cuctinomycin, carubicin, carzinophilin, chromomycines, clindamycin, ductinomycin, daunorubicin, 6-diazo-5-oxn-1-norieucin, doxorubicin, epirubicin, mitomycins, mycophenolsaure, mogalumycin, olivomycin, peplomycin, plicamycin, porfiromycin, puromycin, streptozocin, tubercidin, ubenimex, zinostatin, zorubicin, aminoglycosides, polyenes, macrolid-antibiotics derivatives and any combinations thereof.

An active agent such as an antialopecia agent is for example selected from, but not limited to, the group comprising minoxidil, cioteronel, diphencyprone and finasteride and any combinations thereof.

An active agent such as an antiacne agent is for example selected from, but not limited to, the group comprising retinoids such as tertionin, isotretinoin, adapalene, algestone, acetophenide, azelaic acid. benzoyl peroxide, cioteronel, cyproterone, mortinide, resorcinol, tazarotene, tioxolone as well as an combinations thereof.

An active agent such as an antipsoriatics agent is for example selected from, but not limited to, the group comprising dithranol, acitretin, ammonium salicylate, anthralin, 6-azauridine, bergapten, calcipotriene, chrysarobin, etritrenate, ionapalene, maxacalcitol, pyrogallol, tacalcitol and tazarotene as well as any combinations thereof.

An active agent such as an immusuppressive agent is for example selected from, but not limited to, the group comprising tacrolimus, cyclosporine, sirolimus, alemtuzumab, azathioprine, basiliximab, brequinar, daclizumab, gusperimus, 6-mercaptopurine, mizoribine, muromonab CD3, pimecrolimus, rapamycin and any combinations thereof.

An active agent such a synthetic mosquito repellent is for example selected from but not limited to the group comprising N,N-diethyl-meta-toluamide (DEET), NN-diethyl benzamide, 2,5-dimethyl-2,5-hexanediolbenzil, benzyl benzoate, 2,3,4,5-bis(butyl-2-ene)tetrahydrofurfural (MGK repellent 11), butoxypolypropylene glycol, N-butylacetanilide, normal-butyl-6,6-dimethyl-5,6-dihydro-1,4-pyrone-2-carboxylate (Indalone), dibutyl adipate, dibutyl phthalate, di-normal-butyl succinate (Tabatrex), dimethyl carbate (endo, endo)-dimethyl bicycle[2.2.1]hept-5-ene-2,3-dicarboxylate), dimethyl phthalate, 2-ethyl-2-butyl-1,3-propanediol, 2-ethyl-1,3-hexanediol (Rutgers 612), di-normal-propyl isocinchomeronate (MGK Repellent 326), 2-phenylcyclohexanol, p-methane-3,8-diol, and normal-propyl N,N-diethylsucinamate and derivatives or combinations thereof.

An active agent such as a natural insect repellent is for example selected from, but not limited to, the group of dihydronepetalactone, eucalyptus-derived p-menthan-3,8-diol (PMD) repellent, E-9-octadecenoic acid-derived compounds, extracts from limonene, citronella, eugenol, (+) eucamalol (1), (−)-1-epi-eucamalol, or a crude extract from plants such as eucalyptus maculate, vitex rotundifolia, or cymbopogan, maltitol compound, peppermint oil, cinnamon oil, and nepetalaclone oil, azadirachitin, other neem derived compounds and combinations thereof.

Another group of active agents are for example vaccines such as inactivated vaccines, recombinant protein vaccines, live-attenuated vaccines, viral vector (adenovirus) vaccines, DNA vaccines, mRNA vaccines or a combination thereof. A particle of the present invention comprising a vaccine optionally further comprises an adjuvant. The adjuvant is for example an aluminum salt based adjuvant such as crystalline aluminum oxyhydroxide, aluminum hydroxide, aluminum phosphate, Imject™ Alum, which is a mixture of aluminum hydroxide, magnesium hydroxide or a combination thereof; an emulsion adjuvant, a toll-like receptor (TLR) agonist, a protein carrier such as KLH, a small peptide or a combination thereof.

An active agent such as a neurologic drug is for example selected from, but not limited to, the group of mexiletine, nusinersen, valproic acid, phenobarbital, primidone, benzodiazepines, clobazam, clonazepam, diazepam, midazolam, carbamazepine, eslicarbazepine, ethosuximide, felbamate, gabapentin, hydantoin, fosphenytoin, phenytoin, lacosamide, lamotrigine, levitracetam, oxcarbazepine, perempanel, pregabalin, retigabine, rufinamide, stiripentol, tetracosactide, tiagabine, topiramate, vigabatrin, zonisamide, antimigraine, homeopathy, oligotherapy, ergot alkaloids, methyergide, antiepileptic (e.g., topiramate), antiserotonergic (e.g., flunarizine, oxetorone, pizotifen), beta-blockers (e.g., flunarizine, oxetorone, pizotifen), beta-blockers (e.g., pizotifen), beta-blockers (e.g., pizotifen), metoprolol, propranolol), acetylsalicylic acid, caffeine, paracetamol, acetylsalicylic acid, metoclopramide, almotriptan, eletriptan, frovatriptan, naratriptan, rizatriptan, sumatriptan, zolmitriptan, dihydroergotamine, ergotamine, ibuprofen, ketoprofen, antimyasthenics, anticholinesterases, eculizumab, spironolactone, amifampridine, antiparkinsonian, anticholinergic, dopaminergic, apomorphine, bromocriptine, piribedil, pramipexole, ropinirole, rotigotine, amantadine, entacapone, tolcapone, levodopa, baclofen, dantrolene, piracetam, tizanidine, acetyl-leucine, betahistine, meclozine, piracetam, acetazolamide, nimodipine, oxitriptan, moxisylyte, pentoxifylline, piracetam, Vinburnine, vincamine, amitriptylline, clomipramine, imipramine, carbamazepine, gabapentin, pregabalin, capsaicin, lidocaine, gabapentin, carbamazepine, phenytoin, tetrabenazine, memantine, donepezil, galantamine, rivastigmine, rivastigmine, methylphenidate, modafinil, pitolisant, sodium 4-hydroxybutyrate, idebenone, inotersen, patisiran, tafamidis, alemtuzumab, biotin, cladribine, dimethyl fumarate, fampridine, fingolimod, glatiramer acetate, interferons, mitoxantrone, natalizumab, ocrelizumab, teriflunomide, riluzole, dopaminergic agonists, pramipexole, ropinirole, rotigotine, opioids, oxycodone, naloxone, sultiam, botulinum toxin and combinations thereof.

The polymer, gel, particle, composition or film of the present invention is for example used in a method of preventing and/or treating a viral infection such as Covid, e.g., Covid-19, influenza, or hepatitis such as hepatitis A, B, C, D or E. The vaccine is for example a spike protein such as the spike protein of a corona virus, e.g., SARS-COV2 which has for example a MW of 135 kDa. Another vaccine is for example a cell receptor or a part thereof such as a binding motif for a virus or bacterium. Such binding motif of a cell receptor for example interacting with a corona virus, e.g., SARS-COV2 is for example a receptor binding motif (part N-Nter): GNYNYLYRLFRKSNLKPFERDISTEIYQAGSTPC (SEQ ID NO.1; e.g., 34aa, 4 kDa or a receptor binding motif (part C-Nter): CYFPLQSYGFQPTNGVGYQPYR (SEQ ID NO.2; 22aa, 2.6 kDa).

An active agent such as an antidiabetic drugs is for example selected from, but not limited to, acetazolamide, dulaglutide, exenatide, liraglutide, semaglutide, metformin, glibenclamide, saxagliptin, sitagliptin, vildagliptin, biguanides, saxagliptin, sitagliptin, vildagliptin, acarbose, miglitol, glinides, repaglinide, glibenclamide, gliclazide, glimepiride, glipizide, liraglutide, xultophy, diazoxide, glucagon, alirocumab, evolocumab, omacor, fatty polyunsaturated acids, lomitapide, vitamin E, bezafibrate, ciprofibrate, fenofibrate, gemfibrozil, ezetimibe, atorvastatin, fluvastatin, pravastatin, rosuvastatin, simvastatin, amlodipine, ezetimibe, volanesorsen,

The present invention is also applicable to other anti-diabetic drugs and anti-obesity drugs, including but not limited to leptin such as metreleptin (Myalept), glucagon suppressor, glucagon receptor antagonists, amylin (e.g., Pramlintide (AC0137, AC137, triPro-amylin), anti-ghrelin, amylin agonists, calcitonin such as salmon calcitonin, calcitonin agonists, extenatide, dual amylin calcitonin receptor agonists (DACRA), analogs or combinations thereof.

An active agent such as an anticancer drug is for example selected from, but not limited to, the group of atezolizumab, avelumab, bevacizumab, blinatumomab, catumaxomab, cemiplimab, cetuximab, daratumumumab, dinutuximab beta, durvalumab, ibritumomab tiuxetan, ipilimumab, nivolumab, obinutuzumab, ofatumumumab, panitumumumab, pembrolizumab, pertuzumab, ramucirumab, rituximab, siltuximab, trastuzumab, brentuximab vedotine, gemtuzumab ozogamicin, inotuzumab ozogamicin, trastuzumab emtansine, tretinoin, bexarotene, methoxsalene, porfimer, methyl aminolevulinate, lenalidomide, pomalidomide, thalidomide, arsenic trioxide, asparaginase, crisantaspase, aflibercept, panobinostat, sonidegib, vismodegib, niraparib, olaparib, talazoparib, venetoclax, afatinib, everolimus, idelalisib, binimetinib, cobimetinib, trametinib, dabrafenib, encorafenib, vemurafenib, abemaciciclib, palbociclib, alectinib, axitinib, AZD9291, bosutinib, brigatinib, cabozantinib, ceritinib, crizotinib, dasatinib, erlotinib, gefitinib, ibrutinib, imatinib, lapatinib, lenvatinib, nilotinib, osimertinib, pazopanib, ponatinib, regorafenib, ribociclib, ruxolitinib, sorafenib, sunitinib, vandetanib, midostaurin, temsirolimus, bortezomib, carfilzomib, ixazomib, alkylsulfonates, busulfan, altretamine, dacarbazine, estramustine, mitomycin, pipobroman, procarbazine, temozolomide, thiotepa, trabectedine, carboplatin, cisplatin, oxaliplatin, bendamustine, chlorambucil, chlormethine, cyclophosphamide, ifosfamide, melphalan, nitrosoureas, carmustine, fotemustine, lomustine, streptozocine, taxanes, methotrexate, pemetrexed, raltitrexed, cladribine, clofarabine, fludarabine, mercaptopurine, nelarabine, pentostatin, thioguanine, azacitidine, capecitabine, cytarabine, decitabine, fluorouracil, gemcitabine, cytarabine, daunorubicin, camptothecin and derivatives, irinotecan, topotecan, daunorubicin, doxorubicin, epirubicin, idarubicin, mitoxantrone, pixantrone, podophyllotoxin and derivatives, degarelix, abiraterone, apalutamide, bicalutamide, cyproterone, enzalutamide, flutamide, nilutamide, fulvestrant, tamoxifen, toremifene, anastrozole, exemestane, letrozole, buserelin, goserelin, leuprorelin, triptorelin, lanreotide, octreotide, estrogens, progestins, somatostatin, cytokines, interferons, interleukin, axicabtagen ciloleucel, tisagenlecleucel and combinations thereof. An example for a peptide active agent is insulin, incretin or their analogues. Insulin, incretin or their analogues according to the present invention refers to human or non-human, recombinant, purified or synthetic insulin, incretin, insulin or incretin analogues. Insulin is a peptide hormone secreted by the pancreas, isolated from a natural source or made by genetically altered microorganisms or produced synthetically including synthetic human insulin, synthetic bovine insulin, synthetic porcine insulin, synthetic whale insulin, and metal complexes of insulin, such as zinc complexes of insulin, protamine zinc insulin, and globin zinc. As used herein, “non-human insulin” is the same as human insulin but from an animal source such as pig or cow or any other animal. An insulin analogue according to the present invention is an altered insulin, different from the insulin secreted by the pancreas, but still available to the body for performing the same action as natural insulin. Through genetic engineering of the underlying DNA, the amino acid sequence of insulin can be changed to alter its ADME (absorption, distribution, metabolism, and excretion) characteristics. Examples include insulin lispro, insulin glargine, insulin aspart, insulin glulisine, insulin detemir, humulin, degludec, Gla-300. The insulin can also be modified chemically, for example, by acetylation. An insulin analogue is for example an altered insulin which is able to perform the same action as insulin.

Natural insulin is for example derived from a preproinsulin protein which is secreted in the body with A-chain, C-peptide, B-chain, and a signal sequence. Initially, the signal sequence is removed leaving the remaining A-chain, C-peptide and B-chain, also termed “proinsulin”. After the C-Peptide is cut off, the A-chain and B-chain are left to form insulin.

Insulin according to the present invention includes rapid-acting insulin, very rapid-acting insulin, intermediate-acting insulin, and long-acting insulin. Non-limiting examples of rapid-acting insulin are lyspro insulin (Lysine-Proline insulin, e.g., sold by Eli Lilly as Humalog™), glu-lysine insulin (e.g., sold by Sanofi-Aventis as Apidra™), Actrapid™ and NovoRapid™ (both available from Novo Nordisk), aspart insulin (e.g., sold by Novo Nordisk as Novolog™). A non-limiting example of very rapid-acting insulin is Viaject™. Non-limiting examples of intermediate-acting insulin are NPH (e.g., Neutral Protamine Hagedorn) and Lente insulin. A non-limiting example of long-acting insulin is Lantus™ (insulin glargine). In some preferred embodiments, the insulin is Insugen™ e.g., from Biocon™. Insulin also includes a mixture of different types of insulin. Some non-limiting examples of a such a mixture are Mixtard®30, Mixtard®40, and Mixtard®50, which are mixtures of different proportions of short-acting insulin and NPH (intermediate duration) insulin.

Incretins are a group of metabolic hormones that stimulate a decrease in blood glucose levels. Incretins are released after eating and augment the secretion of insulin released from pancreatic beta cells of the islets of Langerhans by a blood-glucose—dependent mechanism.

Some incretins (GLP-1) also inhibit glucagon release from the alpha cells of the islets of Langerhans. In addition, they slow the rate of absorption of nutrients into the blood stream by reducing gastric emptying and may directly reduce food intake. The two main candidate molecules that fulfill criteria for an incretin are the intestinal peptides glucagon-like peptide-1 (GLP-1) and gastric inhibitory peptide (GIP, also known as: glucose-dependent insulinotropic polypeptide). Both GLP-1 and GIP are rapidly inactivated by the enzyme dipeptidyl peptidase-4 (DPP-4). Both GLP-1 and GIP are members of the glucagon peptide superfamily. The incretins are natural or synthetic incretins or a combination thereof.

An incretin analogue according to the present invention is an altered incretin, different from the incretin secreted by the body, but still available to the body for performing the same action as natural incretin. Through genetic engineering of the underlying DNA, the amino acid sequence of incretin can be changed to alter its ADME (absorption, distribution, metabolism, and excretion) characteristics.

Another active agent of the present invention is for example a glucagon-like peptide (GLP-1) agonist and its analogues such as exenatide, lixisenatide (CAS no. 320367-13-3), liraglutide (CAS no. 204656-20-2), exendin-9 (CAS no. 133514-43-9), AC3174 ([Leu(14)]exendin-4, e.g., Amylin Pharmaceuticals, Inc), taspoglutide (CAS no. 275371-94-3), albiglutide (CAS no. 782500-75-8), semaglutide (CAS no. 910463-68-2), LY2189265 (dulaglutide™; CAS no. 923950-08-7), and CJC-1134-PC (a modified Exendin-4 analogue conjugated to recombinant human albumin, e.g., ConjuChem™).

Insulin, GLP-1 agonist and their analogues include for example derivatives that are modified (i.e., by the covalent attachment of a non-amino acid residue to the protein). For example the protein includes proteins that have been modified, e.g., by glycosylation, acetylation, PEGylation, phosphorylation, amidation, or derivatization by known protecting/blocking groups. Optionally high-MW PEG is attached to the proteins with or without a multifunctional linker either through site-specific conjugation of the PEG to the N- or C-terminus thereof or via epsilon-amino groups present on lysine residues. Additionally, the derivative may contain one or more non-classical amino acids, for example D-isomers of the common amino acids, 2,4-diaminobutyric acid, alpha-amino isobutyric acid, A-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Ca-methyl amino acids, and Na-methyl amino acids.

Glucagon and its analogue is for example selected from the group consisting of Glucagen (Novo Nordisk), GlucaGen kit (Novo Nordisk), Basqsimi (Eli Lily).

Leptin and its analogues is for example selected from the group consisting of Human Leptin expressed for example in E. coli, LEP 24P Porcine (Biorbyt), Myalepta (Aegerion Pharmaceuticals), Metreleptine (Aegerion Pharmaceuticals).

For example, insulin is loaded in the particle in an amount of 0.01 IU/mg up to 20 IU/mg, leptin is loaded in an amount of 0.01 μg/mg up to 800 μg/mg, or exenatide is loaded in an amount of 0.01 μg/mg up to 800 μg/mg. The insulin is for example selected from the group consisting of human insulin, lantus, lispro, novorapid, glulisine, humulin, regular, degludec, NPH, aspart, Gla-300, glargine, detemir, mixed insulin or a combination thereof.

Insulin-encapsulated nanoparticles of the present invention are for example indicated as SLIM particle in the present invention. SLIM is the acronym of: “Sub-Lingual-Insulin-Modality” where two different types AF and BF exist. AF-type corresponds to a SLIM particle comprising human insulin conceived for a slow release profile. A SLIM-AF nanoparticle is composed of multiple layers that releases insulin stepwise following the degradation of the particle layer by layer. BF-type corresponds to a SLIM particle comprising human insulin, but is conceived for a fast release profile. It is a porous nanoparticle loaded with human insulin. The pores of the particle filled with insulin act as a reservoir which releases insulin in a continuous flow once the particles reach the blood stream.

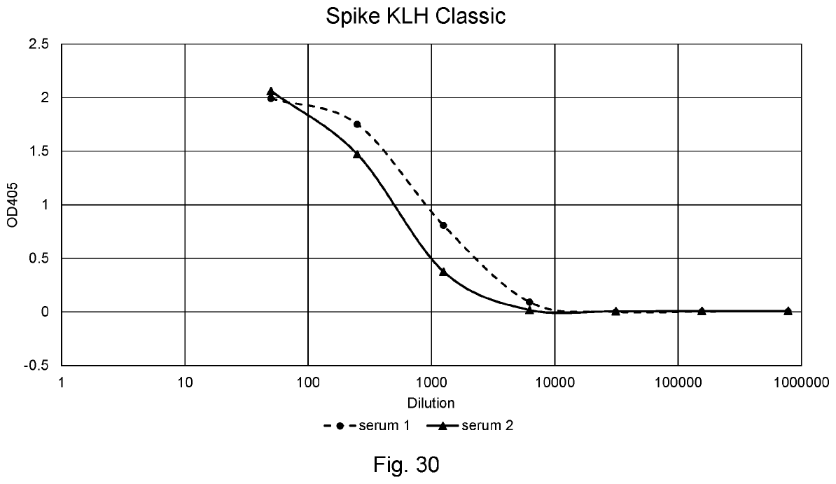

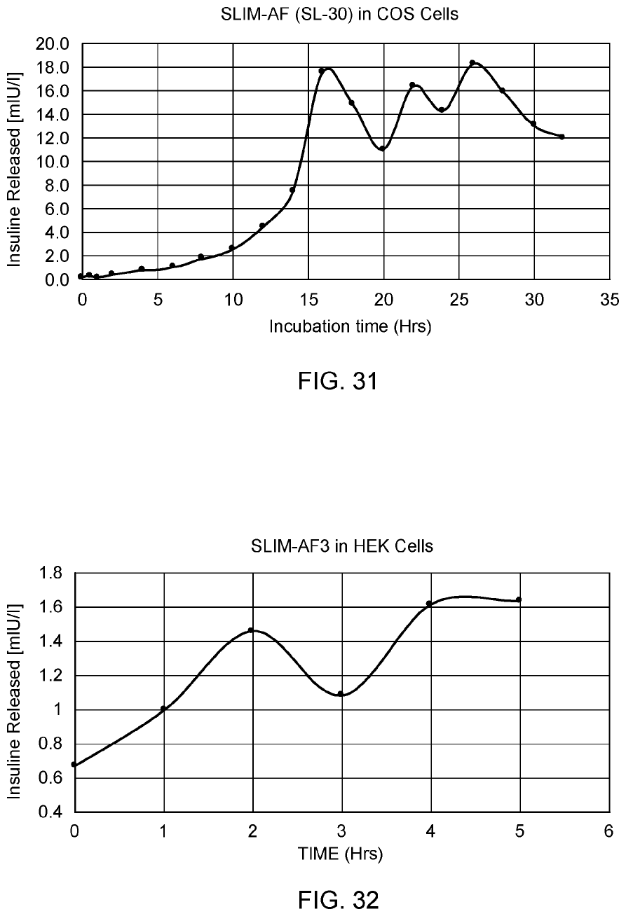

FIG. 31 shows for example a nanoparticle (NP) of the present invention of slow release, not all the NP of slow release have exactly the same profile of release as shown in this Fig. as not all NP of fast release have the same profile of release as shown for example in FIG. 32. The parameters that determine the profile exactly are the particle size, the number of layer and the dose of the active agent such as insulin in every layer.

The size of a pore in the particle of the present invention is for example in a range of about 10 to about 500 Å, about 25 to about 400 Å, about 50 to about 350 Å, about 100 to about 250 Å or about 150 to about 200 Å.

The polymer, gel or particle of the present invention comprises one or more active agents. The choice of an additional active agent depends for example on the use of the polymer, gel or particle in a specific treatment. This is for example treatment of gestational diabetes (e.g., pregnancy diabetes), which is accompanied by lipid peroxidation. The following antioxidants are considered as active agents for application or co-application together with insulin or an analogue: glutathione, glutathione peroxidase and vitamins, including, folic acid, vitamin E, a seleno-amino acid or a combination thereof.

Another example is the use of the polymer, gel or particle in treating Type II diabetes related to obesity, which is accompanied with excess activity of cytokines and kidney oxidative stress. One or more of the following antioxidants are active agents of the particle, which are administered or co-administered together with insulin or an analogue: organic salts of Zn, omega-3 and SOD. Additionally, at least one free amino acid and/or biotin may be added to composition for treating Type II diabetes related to obesity.

A further example is the use of a polymer, gel or particle for the treatment of Type I diabetes which is accompanied by an amino acid misbalance. One or more of the following compounds are active agents of the polymer, gel or particle, which are administered or co-administered together with insulin or an analogue: amino acids, antioxidants such as: vitamin K and/or organic salts of Zn, organic salts of chrome, a seleno-amino acid and cofactors such as vitamins of group B (e.g., to help nervous system and neurotransmitters formation), including, but not limited to, any one or more of vitamin B1 (thiamine), vitamin B2 (riboflavin), vitamin B3 (niacin or niacinamide), vitamin B5 (pantothenic acid), vitamin B6 (pyridoxine, pyridoxal, or pyridoxamine, or pyridoxine hydrochloride), vitamin B7 (biotin), vitamin B9 (folic acid), vitamin B 12 (various cobalamins), vitamin B complex and combinations thereof.

For example, for the treatment of a metabolic disorder/disease, pectin and/or amylin can be added to the polymer, gel or particle and/or composition comprising insulin, proinsulin and/or C-peptide.

The weight ratio of particles to active agent such as insulin or its analogue, glucagon suppressor or its analog, or a combination thereof is for example within the range of 100:1 to 1:1, within the range of 75:1 to 25:1 or within the range of 20:1 to 3:1. Alternatively, the weight ratio of particles to active agent such as proinsulin is for example within the range of 200:1 to 2:1, within the range of 150:1 to 50:1 or within the range of 30:1 to 6:1. Alternatively, the weight ratio of particles to active agent such as C-peptide is for example within the range of 200:1 to 1:1, within the range of 200:1 to 2:1 or within the range of 40:1 to 6:1.

The polymer, gel, particle, composition or film of the present invention is for example used as a medicament. It is for example used in a method of preventing and/or treating a metabolic disorder/disease such as hyperlipidemia, hypercholesterolemia, hyperglyceridemia, hyperglycemia, insulin resistance, obesity, hepatic steatosis, kidney disease, fatty liver disease, non-alcoholic steatohepatitis, a respiratory disease, an inflammatory disease, obesity, a neuronal disease, a viral disease, a cancer disease, a disease of the central nervous system, a cardio-vascular disease or a combination thereof.

The film is for example an orodispersible film. The polymer, gel, particle, composition or film is administered locally or systemically for example orally, sublingually, buccally, intravenously, subcutaneously, intramuscularily, enterally, parenterally, topically, vaginally, rectally, intraocularily or a combination thereof. The orodispersible film consists of or comprises for example, but is not limited to, hydroxypropylmethylcellulose, polyvinylpyrrolidone, polyvinyl alcohol or a combination thereof. Alternatively, the film of the present invention is made of the polymer of the present invention.

The orodispersible film of the present invention comprises for example one or more nanoparticles, microparticles or a combination thereof. The particle is for example printed onto the orodispersible film. The particles comprise the same or different active agents. The particles comprise the active agent in the same or different amounts. The orodispersible film is used in personalized medicine. For example it is loaded with different particles comprising the daily doses of an active agent that has to be administered to a patient for example every day.

The film comprises or consists of for example, but is not limited to, hydroxypropylmethylcellulose, polyvinylpyrrolidone, polyvinyl alcohol or a combination thereof.

A particle of the present invention optionally comprises a coating such as a surface coating e.g., 3-aminopropyltriethoxysilane, 3-aminopropyl-trimethoxysilane, carboxyl containing molecules such as 5-(triethoxysilyl)pentanoic acid, epoxy containing molecules such as 3-glycidyloxypropyl-trimethoxysilane, thiol containing molecules such as 3-mercaptopropyl-trimethoxysilane or 3-mercaptopropyl-triethoxysilane or maleimide containing molecules such as m-maleimidobenzolt-N-hydroxysuccinimide ester, or N-(p-maleimidophenyl)isocyanate, or a combination thereof. The coating enhances for example the transport of the particle across the mucosa such as oral mucosa, buccal mucosa, sublingual mucosa, rectal mucosa, vaginal mucosa, mucosa of the eye, nasal passages, mouth and lip area or the external ear.

The total surface area of the oral mucosal lining in a human for example is approximately 100 cm². The oral mucosa can be divided into the following 3 types: buccal mucosa, sublingual mucosa and palatal mucosa. Individual types of mucosa anatomically vary in their thickness, degree of the epithelium keratinization, and hence the permeability for drugs, particles and other physiologically active agents. These mucosal categories also differ significantly in their structure including the proportions of the immune cell types.

Significant external factors influencing the penetration of the polymer, gel or particle through the mucosa include the continuous production of saliva providing washing of the mucosal surface and the formation of a thin film and the movement of the oral mucosa and tongue during speaking, eating, drinking and chewing.

Given the similarity of the structure and degree of keratinization of mucosa with humans, the pig is currently the most widely used model animal for monitoring the transfer of substances and particles through the oral mucosa (both in-vivo and ex-vivo experiments; e.g., Marianne O. Larsen and Bidda Rolin, ILAR Journal, Vol. 45, Issue 3, 2004, p. 303-313; A J F King, British journal of Pharmacology 1666, p. 877-894, 2012; M Jensen-Waern et al., Laboratory Animals 2009; 43: p. 249-254; Gabel H, et al., Horm Metab Res. 1985 June; 17(6): p. 275-80; Strauss et al. Diabetology & Metabolic Syndrome 2012, 4:7).

Particles carrying mucosal vaccines often lack an effect on immune cells and the immune system, respectively. Moreover, some particles are insufficient in penetration and transition of the mucosa, especially in model animal species.

Given the barriers and physiological conditions in the oral cavity, the active agents need to be specially prepared and administered by appropriate administration forms. Standard oral drug formulations include buccal and sublingual tablets, pastilles, sublingual sprays, oral gels and solutions. However, these drug forms do not allow the ingestion of food or drink, and in the case of sublingual sprays even during speaking. These formulations are preferred for dealing with the administration of low-molecular substances and insulin. More advanced mucoadhesive drug forms can include solutions (which form a viscous gel directly on the mucosa), sublingual effervescent tablets and mucoadhesive buccal and sublingual films and sheets, respectively.

Different types of particles of the present invention can be prepared which differ in the structure. Such particles are for example multi-layer particles (A-type; Table 1) or porous particles (B-type; Table 2) comprising different concentrations of an active agent such as an anti-diabetic drug or combinations thereof for example insulin:

TABLE 1
shows multi-layer SLIM particles comprising different concentrations
of insulin such as recombinant human insulin.

| | | Concentration of Insulin | |
|---|---|---|---|
| | | per milligrams of particles | |
| # | Sample | (IU/mg) | |
| 1 | Multi-layers particles | 1.36 | IU/mG |
| 2 | Multi-layers particles | 0.24 | IU/mG |
| 3 | Multi-layers particles | 0.64 | IU/mG |
| 4 | Multi-layers particles | 2 | IU/mG |
| 5 | Multi-layers particles | 1.46 | IU/mG |
| 6 | Multi-layers particles | 3.8 | IU/mG |
| 7 | Multi-layers particles | 1.46 | IU/mG |
| 8 | Multi-layers particles | 0.1 | IU/mG |
| 9 | Multi-layers particles | 5 | IU/mG |
| 10 | Multi-layers particles | 10 | IU/mG |

TABLE 2
shows porous particles comprising different
concentrations of insulin.

| | | Concentration of Insulin | |
|---|---|---|---|
| | | per milligrams of particles | |
| # | Sample | (IU/mg) | |
| 1 | Porous particles | 0.2 | IU/mG |
| 2 | Porous particles | 1.92 | IU/mG |
| 3 | Porous particles | 2.16 | IU/mG |
| 4 | Porous particles | 3.0 | IU/mG |
| 5 | Porous particles | 1.8 | IU/mG |
| 6 | Porous particles | 1.33 | IU/mG |
| 7 | Porous particles | 0.58 | IU/mG |
| 8 | Porous particles | 0.92 | IU/mG |
| 9 | Porous particles | 1.26 | IU/mG |
| 10 | Porous particles | 0.69 | IU/mG |
| 11 | Porous particles | 1.22 | IU/mG |

The release of the active agent from the polymer, gel or particle of the present invention is for example a monophasic or multiphasic release.

A polymer, gel or particle of the present invention is for example used in personalized medicine, wherein the active agent is for example resorbed via the mucosa. Polymers, gels or particles comprising different active agents and/or an active agent in different concentrations and amounts, respectively, are for example dotted or printed on a film. The polycondensation catalyst for the preparation of a hybrid particle is for example an acid or basic catalyst. Preferably, it is a basic catalyst, such as NaOH, KOH, LiOH, Mg(OH)₂, NH₄OH, a basic peptide, a basic amino acid for production methods using silicon alkoxide, a basic peptide, N,N′-dimethylethylenediamine or a combination thereof. Alternatively, it is an acid catalyst, such as nitric acid (HNO₃) for production methods using a titanium alkoxide; in this case it is generally an aqueous nitric acid solution at 0.01 Mol/L.

The active agent is added to the saturated carbon donor and/or its mixture with a metal oxide precursor, a metal oxide or a combination thereof in the method of production of the present invention. The active agent is for example in pure form, a liquid or solid form, dissolved in an hydro-alcoholic solution, dissolved in a water-organic solvent or a combination thereof. The active agent is for example incorporated into the particle and/or in the pore.

The composition comprising the polymer, gel or particle further comprises for example a excipient such as a pharmaceutically acceptable excipient, a cosmetic acceptable excipient, an agricultural acceptable excipient or a combination thereof.

The excipient is for example useful for the improvement of the therapeutic effect of the active agent and others influencing active agent consistence and the final dosage form. Suitable excipients include: Antifoaming agents (e.g. dimethicone, simethicone); Antimicrobial preservatives (e.g. benzalkonium chloride, benzelthonium chloride, butylparaben, cetylpyridinium chloride, chlorobutanol, chlorocresol, cresol, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol); Chelating agents (e.g. edetate disodium, ethylenediaminetetraacetic acid and salts, edetic acid); Coating agents (e.g. sodium carboxymethyl-cellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcrystalline wax, zein); Colorants (e.g. caramel, red, yellow, black or blends, ferric oxide); Complexing agents (e.g. ethylenediaminetetraacetic acid and salts (EDTA), edetic acid, gentisic acid ethanolmaide, oxyquinoline sulfate); Desiccants (e.g. calcium chloride, calcium sulfate); Flavors and perfumes (e.g. anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); Humectants (glycerin, hexylene glycol, propylene glycol, sorbitol); Polymers (e.g., cellulose acetate, alkyl celluloses, hydroxyalkylcelluloses, acrylic polymers and copolymers); Sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); This list is not meant to be exclusive, but instead merely representative of the classes of excipients and the particular excipients which may be used in oral dosage compositions of the present invention.

Alternatively or in addition, the polymer, gel, particle, composition or film of the present invention comprises hypericin, dotarem, Fe₂O₃, Cynanine5.5 (Cy5.5), tetrakis(hydroxymethyl)phosphonium chloride (THPC), Dyomic DY-700, (TrpyRu) (Terpyridine)2 Ruthenium II trichloride, TrpyOs (Terpyridine)₂ Osmium II Trichloride, 2-propenyl-N-acetyl-neuraminic acid (GNP), Gadolinum (Gd), Mangan (Mn), laccase, rhodamine B, Oregon Green, lndocyanine green (ICG) active dye, their analogs and derivatives or a combination thereof.

Optionally the polymer, gel, particle, composition or film of the present invention comprises a release rate modulating agent for example selected from, but not limited to, the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl [beta]-D-glucopyranoside; n-decyl [beta]-D-maltopyranoside; n-dodecyl [beta].-D-glucopyranoside; n-dodecyl [beta]-D-maltoside; heptanoyl-N-methylglucamide; n-heptyl-.beta.-D-glucopyranoside; n-heptyl [beta]-D-thioglucoside; n-hexyl [beta]-D-glucopyranoside; nonanoyl-N-methylglucamide; n-noyl[beta].-D-glucopyranoside; octanoyl-N-methylglucamide; n-octyl-. [beta]-D-glucopyranoside; octyl [beta].-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, Ci2-I5 dimethyl hydroxyethyl ammonium chloride, Ci2-I 5 dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide; lauryl dimethyl (ethenoxy)4 ammonium chloride, lauryl dimethyl (ethenoxy)4 ammonium bromide, N-alkyl (C]2-I s) dimethylbenzyl ammonium chloride, N-alkyl (Ci4-is)dimethyl-benzyl ammonium chloride, N-tetradecyldimethylbenzyl ammonium chloride monohydrate, dimethyl didecyl ammonium chloride, N-alkyl and (Ci2-I4) dimethyl 1 -napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(Ci2-i4) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, Ci2 trimethyl ammonium bromides, Ci5 trimethyl ammonium bromides, C7 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylamrhonium bromide, dodecyltriethylammonium-bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar and combinations thereof.

Optionally the polymer, gel, particle, composition or film of the present invention may further contain hydrophilic solvents, lipophilic solvents, humectants/plasticizers, thickening polymers, surfactants/emulsifiers, fragrances, preservatives, chelating agents, UV absorbers/filters, antioxidants, keratolytic agents, dihydroxyacetone, penetration enhancers, dispersing agents or deagglomerating agents as well as mixtures thereof.

Optionally a cosmetic composition further comprises one or more anti-ageing agents, sunblocking agents, antiwrinkle agents, moisturizing agents, anti-dandruff agents especially selenium sulfide, vitamins, saccharides, oligosaccharides, hydrolysed or non-hydrolysed, modified or unmodified polysaccharides, amino acids, oligopeptides, peptides, hydrolysed or non-hydrolysed, polyamino acids, enzymes, branched or unbranched fatty acids and fatty alcohols, animal, plant or mineral waxes, ceramides and pseudoceramides, hydroxylated organic acids, antioxidants and free-radical scavengers, chelating agents, seborrhoea regulators, calmants, cationic surfactants, cationic polymers, amphoteric polymers, organomodified silicones, mineral, plant or animal oils, polyisobutenes and poly[alpha]-olefins), fatty esters, anionic polymers in dissolved or dispersed form, nonionic polymers in dissolved or dispersed form, reducing agents, hair dyes or pigments, antioxidants, free radical scavengers, melanoregulators, tanning accelerators, depigmenting agents, skin-coloring agents, liporegulators, thinning agents, antiseborrhoeic agents, anti-UV agents, keratolytic agents, refreshing agents, cicatrizing agents, vascular protectors, antiperspirants, deodorants, skin conditioners, immunomodulators, nutrients and essential oils and perfumes, substance having a hair-care activity, agents for combating hair loss, hair dyes, hair bleaches, reducing agents for permanent waves, hair conditioners, nutrients or combinations thereof.

In the following some variations of the method for producing a polymer or particle are further specified:

The method for the production of the particle comprising for example the steps of:
a) preparing a saturated solution of a carbon donor such as a saccharide in a water/alcohol or a water/alcohol/organic solvent to prepare a saturated solution, wherein the ratio of water to alcohol or the ratio of water to alcohol and organic compound is from about 20:80 to about 80:20, from about 40:60 to about 60:40 or from about 45:55 to about 55:45,
b) optionally filtrating the saturated solution to eliminate insoluble particulates,
c) mixing the saturated solution of step a) or b) with a metal oxide precursor such as tetraethoxysilane (TEOS) or tetra-methyl-ortho-silicate (TMOS), or with an alcoholic solution of a metal oxide precursor such as a silica precursor, wherein the ratio of metal oxide to saccharide is in a range from about 0,01% to about 99,9%, more preferably from about 25% to about 50%,
d) optionally mixing the saturated solution of step a) or b) with an active agent with or without a carboxyl or hydroxide moiety
e) preparing an alcoholic or hydro-alcoholic solution of a polycondensation catalyst such as NaOH, KOH, NH₄OH, LiOH, Mg(OH)₂, a basic peptide, a basic amino acid, N,N′-dimethylethylenediamine, its analogue or derivative, or a combination thereof and adding it to step a), b) and/or c), and/or d)
f) optionally adding an organic compound with or without a carboxyl or hydroxide moiety to step e),
g) optionally adding a metal, a metalloid or a combination thereof to the solution of step e), wherein the metal is selected from the group consisting of Si, Ti, Fe, Au, Ag, Al, Cu, Cr, Gd, Zn, Zr, Ru, Rh, Pd, Sn, Cd, Sb, Te, U, Er, Yb, ln, Mg, Mn, its salt, its oxide, its alloy or a combination thereof,
h) stirring the mixture for a period of 2 to 72 h, 10 to 48 h or 12 to 24 h, preferably for 24 to 72 hours, more preferably for 48 hours at a temperature between −15° C. and 65° C., preferably between 0° C. and 20° C. for forming the particle,
i) isolating the formed particle and optionally washing the particle to remove unreacted saccharide, saccharide substitute, optionally unreacted organic compound, or unreacted active compounds, or unreacted metal or metalloid, or a combination thereof,
j) optionally repeating steps a) and d) to form two or more layers,
wherein the order of the steps is changed optionally.

A silica precursor is for example tetraethoxysilane (TEOS) or tetra-methyl-ortho-silicate (TMOS), sodium silicate (Na₂SiO₃) in an alcoholic solution under basic conditions or a combination thereof.

The present invention additionally refers to a method for the production of the porous particle for example comprising the steps of:
a) preparing the particle according to the method of the present invention and isolating the particle via centrifugation and/or filtration,
b) washing the particle in water, alcohol or a combination thereof,
c) optionally sonicating the particles for example in water, alcohol or a combination thereof,
d) optionally repeating b) one or more time to remove unreacted saccharide or saccharide substitute from the surface and/or the bulk of the particle,
e) optionally controlling the pore size of the particle,
f) optionally loading the pores of the particles with an active agent selected from the group of natural or synthetic molecule, an enzyme, a protein, a peptide, an antibody, an oligonucleotide, a gene, a gene fragment, an antigen, a vaccine, a cellular organism such as microorganisms, e.g., bacteria, yeasts, fungi, algae, cells of animal or plant origin,
wherein the order of the steps is changed optionally.

The water for washing the particle in step b) of any method of the invention is preferably hot water of for example 30° C. to 95° C., 40° C. to 90° C., 50° C. to 80° C., or 60° C. to 70 ° C. The washing step is prepared very thoroughly. The pore size is for example determined by direct observation under an electron microscope.

Moreover, the present invention is directed to a method for the production of the polymer or particle comprising the steps of:
a) preparing a saturated solution of saccharide, saccharide substitute or a combination thereof in a water/alcohol or a water/alcohol/organic solvent to prepare a saturated solution, wherein the ratio of water to alcohol or the ratio of water to alcohol and organic compound is from about 20:80 to about 80:20, from about 40:60 to 60:40 or from 45:55 to 55:45,
b) optionally filtrating the saturated solution to eliminate insoluble particulates,
c) mixing the saturated solution of step a) or b) with a metal oxide precursor such as tetraethoxysilane (TEOS), tetra-methyl-ortho-silicate (TMOS) or with hydro-alcoholic solution of a metal oxide precursor such, wherein for example the ratio of metal oxide to saccharide, saccharide substitute or combination is in a range from about 10% to about 90%, more preferably from about 25% to about 50%,
d) optionally adding an organic compound with or without carboxyl or hydroxyl groups to the solution of step c),
e) optionally adding a metal, a metal oxide, a metalloid or a combination thereof to the solution of step c),
f) preparing an alcoholic or hydro-alcoholic alkali solution of a polycondensation catalyst such as NaOH, KOH, NH₄OH, LiOH, Mg(OH)₂, a basic peptide, a basic amino acid, N,N′-dimethylethylenediamine, its analogue or derivative, or a combination thereof and adding it to step c) and/or d), wherein the alkali solution is present in an amount from about 5% to about 40%, more preferably from about 10% to about 20% based on the final weight of the composition, and
g) stirring the mixture for a period of 2 to 48 h, 10 to 24 h or 12 to 20 h for forming the polymer,
h) optionally sonicating and/or heating the mixture for 4 to 48 h, more preferably for 10 to 24 h for forming the polymer, wherein the order of the steps is changed optionally.

Optionally an active agent is comprised by the polymer, gel or particle. It is added to the polymer at any step(s) of any method of production of the present invention.

Furthermore, the present invention relates to a method for the production of a gel based on the particles of the present invention comprising the steps:
a) dispersing the particle and/or porous particle prepared according to the present invention in an alcoholic or hydro-alcoholic solution under vigorous sonication,
b) preparing a saturated solution of saccharide, saccharide substitute or a combination thereof
c) mixing the saturated solution of step b) with the particles of step a) in a ratio of 1:1, 1:2 or 1:3,
d) adding a metal oxide precursor such as tetraethoxysilane (TEOS) or tetra-methyl-ortho-silicate (TMOS), or a hydro-alcoholic solution of a metal oxide precursor such as silica precursor,
e) optionally adding an organic compound with or without carboxyl or hydroxyl groups to the mixture,
f) preparing an alcoholic or hydro-alcoholic alkali solution of a polycondensation catalyst such as NaOH, KOH, NH₄OH, LiOH, Mg(OH)₂, a basic peptide, a basic amino acid, N,N′-dimethylethylenediamine, its analogue or derivative, or a combination thereof and adding it to step d), wherein the alkali solution is present in an amount from 5% to about 40%, more preferably from about 10% to about 20% based on the final weight of the composition.
g) stirring the mixture for a period of 2 to 48 h, 10 to 24 h or 12 to 20 h for forming the particles containing gel,
h) optionally sonicating and/or heating the mixture for 4 to 48 h, more preferably 10 to 24 h for forming the gel.

In each of the methods of the present invention the ratio between the solution comprising the inorganic component and the saturated carbon donor such as a saccharide solution may vary from 18:01 to 1:4 (v/v), which is for example a molar ratio of between 0.24 and 9.5. Preferably this molar ratio is between 0.32 and 0.95. Advantageously, in a production method in which the carbon donor such as the carbohydrate is maltose and the precursor of inorganic polymers is TEOS, the ratio is about 0.47.

Alternatively, the ratio of the carbon donor such as the a saccharide and inorganic component including the polycondensation catalyst is between 0.00066 and 0.5 (v/v), advantageously between 0.0066 and 0.33 (v/v), more advantageously between 0.066 and 0.3 (v/v).

The molar ratio between metal oxide precursor or metal oxide:carbohydrate:catalyst:water:alcohol is for example 1:1.5:1.6:0:388 or 1:1.5:16.5:252:311.

For example, the saturated solution of the carbon donor such as a carbohydrate, e.g., a saccharide is dropwise mixed with pure TEOS or TMOS, and/or an alcoholic solution of a metal oxide precursor such as TEOS or TMOS.

The particle of the present invention is for example “non-hybrid, non-mixed”, if it comprises a matrix solely based on one or more saccharide(s) such as monosaccharide, disaccharide, oligosaccharide or polysaccharide or a combination thereof. It does not comprise elements other than saccharide(s) in particular, it does not comprise metals or metalloids, or their alloys. The “non-hybrid, non-mixed” particle is obtained by polycondensation of one or more saccharide(s), one or more oligosaccharide(s), or a mixture of one or more saccharide(s) and one or more oligosaccharide(s), from a saturated solution of one or more saccharide(s) and/or one or more oligosaccharide(s). A “non-hybrid, non-mixed” particle is for example obtained from a saturated solution of one or more monosaccharides and/or one or more oligosaccharides. The saturated solution further comprises a solvent which is water, one or more organic solvents or a mixture of water and organic solvent(s). The solvent or combination of solvents is selected according to the carbohydrate monomer(s) or oligomer(s) used. The organic solvent is preferably a polar alcohol, linear or branched, preferably containing 1 to 30 carbon atoms. The organic solvent is preferably ethanol, which has the advantage of having low toxicity and being miscible with water.

The particle is for example “mixed”, if it also comprises one or more polymeric monomers or one or more polymers or a combination thereof. The particle is for example “hybrid”, if it also comprises one or more metal oxide precursors, metal oxides or metalloid compounds. Therefore, the particle is for example “non-hybrid and mixed” or “hybrid and mixed” if it comprises, or not, one or more metal or metalloid compounds.

The oligosaccharides according to the invention comprise for example monomers chosen from glucose, sucrose, galactose, fructose, mannose and their derivatives. The polysaccharide is for example an oligomer comprising more than 10 carbohydrate monomers, preferably having the formula —[Cₓ(H₂O)_{y)}]_{n.}—(wherein y is equal to x−1 and n>10 ), linear or branched, comprising monomers selected from glucose, galactose, sucrose, fructose, mannose and their derivatives.

For the preparation of the polymer, gel or particle, the organic solvent is for example a pure anhydrous alcohol solution, i.e. comprising less than 0.5% by volume of water, or a hydro-alcoholic solution comprising 10 to 99% by volume of alcohol, preferably 20 to 90% by volume of alcohol, advantageously 40% by volume of alcohol. The organic solvent may also be dimethylsulfoxide (DMSO). This can be an anhydrous dimethylsulphoxide solution, i.e. comprising less than 0.5% by volume of water, but preferably a dimethylsulphoxide solution comprising 10-99% by volume of water, preferably 20-90% by volume of water, advantageously 60% by volume of water.

The concentration of the carbon donor such as a carbohydrate e.g., monosaccharides and oligosaccharides in the saturated solution depends on the nature of the monosaccharide and oligosaccharide, the solvent and its physical properties, the temperature, and possibly the salts (e.g. NaCl, KCl, K₂SO₄) used. The saturated solution contains for example up to 14 Mol/L of carbohydrate monomers and/or oligomers, preferably between 4 and 7 Mol/L, advantageously 5 and 6 Mol/L for saccharide, when it is an aqueous or hydro-alcoholic solution. For an alcoholic solution, the saturated solution comprises up to 0.06 Mol/L (at 50° C.) of monosaccharides and/or oligosaccharides, preferably between 0.01 and 0.05 Mol/L, advantageously between 0.02 and 0.03 Mol/L of saccharide in methanol at 20° C. For a dimethylsulfoxide solution, the concentration comprises for example between 87.64 Mol/L and 292.14 Mol/L of monosaccharides and/or oligosaccharides, advantageously between 90 and 100 Mol/L of saccharide.

The solution saturated with monosaccharides and/or oligosaccharides is optionally filtered to remove any undissolved solid particles. Preferably, the filtration is done through a membrane filter (0.22 μm).

The alcoholic solution in the methods of the present invention is for example based on ethanol, methanol or a combination thereof, and comprises ammonia. The solution is prepared by mixing an aqueous solution of ammonium hydroxide, for example between 20% and 30% NHs, with an alcohol solution, for example pure or anhydrous ethanol. The alcoholic ammonia solution comprises between 1 and 10% v/v of the aqueous ammonium hydroxide solution, preferably between 5 and 7% v/v, advantageously 6.66% v/v, i.e. a molar ratio of 2.84% of the aqueous ammonium hydroxide solution to 28%.

A “non-hybrid, non-mixed” particle such as a nanoparticle is for example obtained by mixing the polycondensation catalyst with the saturated solution of the carbon donor such as monosaccharides and/or oligosaccharides with stirring, e.g., at a temperature of between 5° C. and 65° C. or preferably at 21° C. The saturated solution is for example added drop by drop under stirring to the catalyst solution. For example after one hour of stirring, the solution becomes cloudy and whitish particles appear. Agitation is preferably continued for 12 to 24 hours.

The non-hybrid, non-mixed particles are then collected by any suitable means, for example by centrifugation, and optionally they are cold washed with an organic solvent in which the particles are not very soluble, preferably insoluble.

For the preparation of a non-hybrid, non-mixed particle the saccharide is for example saccharide, the alcoholic solution is a hydro-alcoholic solution of 20% to 60%, 30% to 50%, 20%, 40% or 60% methanol, ethanol, propanol or a combination thereof by volume, and contains 2 to 10 Mol/L, 3 to 8 Mol/L, 4 to 5 Mol/L, 2.7, 3.7, 4.7, 5.7. 6.7 or 7.7 Mol/L of saccharide such as maltose.

A mixed non-hybrid particle of the present invention is for example obtained by the polycondensation of one or more monosaccharides one or more oligosaccharides or mixtures thereof, and one or more organic polysaccharides or one or more organic polymers. The matrix comprises covalent, ionic and/or hydrogen bonds between the polymeric organic monomers or polymers and/or oligosaccharides or their hydrolysis products, e.g., carbohydrate acids and/or osidic acids or polyacids. The monosaccharides are for example selected from glucose, galactose, maltose, sucrose, fructose, mannose, and their derivatives.

Oligosaccharides are for example disaccharides or trisaccharides, preferably maltose. Polysaccharides are for example homoglycans or heteroglycans, such as starch, cellulose or derived keys used in food processing such as methylcellulose or carboxymethylcellulose, or dextran.

The organic polymeric monomer(s) is (are) for example selected from polymerizable acids, e.g. hydroxy acids, amino acids, acrylates, methacrylates or alkylcyanoacrylates, and their derivatives. The monomers are for example allowing the production of polymers of the polyester, polyamino acid or polyacrylate type, preferably polymers of the polylactic, polyacrylate, polycyano- or methacrylate, polylactic-glycolic, polyethylene glycol, polyaminoacid type, even more preferably polymers such as polylactic acid (PLA), poly(-caprolactone (PCL)), poly(alkyl cyanoacrylate) (PACA), poly(glycolic acid) (PGA), poly(lactic acid-co-glycolic acid) (PLGA), or poly(methyl methacrylate) (PMMA) and/or their derivatives.

Organic polymers are for example polymers, homo or co-polymers, linear or branched, natural or synthetic. They may be peptides, proteins, such as albumin, gelatin or collagen, or protein fragments. They can also be polyester polymers, polyaminoacids, copolyamino acids or polyacrylates, advantageously polymers of the polylactic, polyacrylate, poly cyano- or methacrylate, polylactic-glycolic, polyoxyethylene-oxypropylene, polyamino acid type, even more advantageously polymers such as polylactic acid (PLA), poly(-caprolactone (PCL)), poly(alkyl cyanoacrylate)s (PACA), poly(glycolic acid) (PGA), poly(lactic acid-co-glycolic acid) (PLGA), or poly(methyl methacrylate) (PMMA) and/or their derivatives.

The mixed non-hybrid particles are for example obtained from a saturated solution of the carbon donor such as one or more monosaccharides and/or one or more oligosaccharides and/or polysaccharides as described above, from a solution of one or more organic polymeric monomers or organic polymers, in the presence of a polycondensation catalyst as described above.

The mixed non-hybrid particles of the present invention, e.g., saccharide-based particles, have the advantage of being bio-compatible without having to be functionalized on the surface in a “post-production” stage, but also being fully biosoluble. The particles are also rigid, stable, with a significant capacity maintained for the storage of one or more biologically active molecules, molecules from which a prolonged release is possible.

The hybrid particle of the present invention comprises a matrix based on one or more polysaccharides or oligosaccharides or a combination thereof, and one or more metal oxide precursors, metal oxides or metalloid compounds.

Hybrid particles are for example obtained by the method of the present invention comprising polycondensation of one or more monosaccharides, one or more oligosaccharides, polysaccharides or a combination thereof, and one or more inorganic polymer precursors comprising one or more metals or metalloids. Their matrix may comprise covalent, ionic and/or hydrogen bonds between the metal or metalloid compound(s) and the monosaccharide, oligosaccharide monosaccharide and/or polysaccharides or their hydrolysis products (carbohydrate acids and/or osidic acids or polyacids).

Hybrid particles are for example obtained by first mixing the saturated solution of one or more monosaccharides, one or more carbohydrate oligomers and/or one or more polysaccharides with one or more inorganic polymer precursors comprising one or more metals or metalloids, in a suitable solvent or mixture of solvents, preferably in the form of a hydroalcoholic solution at 40% alcohol or preferably at 20% alcohol. Then, after stirring and homogenization, this mixture is added, still with stirring, to a solution of a polycondensation catalyst. The reaction takes place at a temperature between 15° C. and 150° C. or preferably at 21° C., for 12 to 24 hours, preferably for 24 hours. The hybrid particles are then collected, for example by centrifugation, and are washed with alcohol or water, in particular to remove the unreacted carbohydrate fraction. A drying step can optionally be provided to remove all traces of solvents.

Alternatively, hybrid particles are for example obtained by first mixing the saturated solution into one or more monosaccharide, one or more oligosaccharide and/or one or more polysaccharide with the catalyst, then after homogenization by stirring until the solution becomes turbid, which takes e.g., between 1 and 6 hours for the formation of very small carbohydrate particles in suspension, and still under stirring, the mixture of one or more inorganic polymer precursors comprising one or more metals or metalloids is added, preferably in the form of a hydro-alcoholic solution.

The particles of the present invention comprise one or more layer(s) such as a single-layer, double-layer or multiple-layers.

The multi-layer particles comprise for example a matrix and one or more other matrices comprising one or more monosaccharide(s), oligosaccharide(s) and/or polysaccharide(s), one or more organic polymeric monomers or one or more organic polymers, one or more metal oxide precursor, metal oxide or metalloid compounds, or a combination thereof.

The multi-layer particles comprise for example successive layers of the same or different matrices, arranged one on top of the other, or an outer matrix enveloping a multitude of single-layer or multi-layer particles.

The multi-layer particles of the present invention are for example obtained by successively polymerizing, optionally in the presence of a polycondensation catalyst, one or more times, any of the following composition or combination of compositions according to the methods of the present invention:
a saturated solution of one or more monosaccharide(s), oligosaccharide(s), polysaccharide(s) or a combination thereof,
a saturated solution of one or more monosaccharide(s), oligosaccharide(s), polysaccharide(s) or a combination thereof and one or more organic polymeric monomers or one or more organic polymers,
a saturated solution of one or more monosaccharide(s), oligosaccharide(s) polysaccharide(s) or a combination thereof and one or more inorganic polymer precursors comprising one or more metal oxide precursor(s), metal oxide(s) or metalloids,
a saturated solution of one or more monosaccharide(s), oligosaccharide(s), polysaccharide(s) or a combination thereof one or more organic polymeric monomers or one or more organic polymers and one or more inorganic polymer precursors comprising one or more metal oxide precursor(s), metal oxide(s) or metalloids,
one or more organic polymeric monomers or one or more organic polymers and one or more inorganic polymer precursors comprising one or more metal oxide precursor(s), metal oxide(s) or metalloids,
one or more inorganic polymer precursors comprising one or more metal oxide precursor(s) or metalloids,
one or more organic polymeric monomers or one or more organic polymers.

The particle and/or the layer of the particle of the present invention comprise or consist of at least one or more matrices, wherein
the matrix is based on one or more monosaccharides or one or more oligosaccharides,
the matrix is made from maltose, sucrose, fructose, or a mixture of thereof,
the matrix includes one or more organic polymeric monomers or one or more organic polymers,
the matrix includes one or more metal or metalloid compounds which are for example selected from Au, Ag, Fe, Gd, Si, Ti, Zn, Zr, its salt, its oxide, its alloy or a combination thereof,
the matrix includes one or more monosaccharides and/or oligosaccharides, one or more organic monomers, one or more organic polymers, one or more metals or metalloids or a combination thereof.

The multi-layer particles of the present invention comprise for example a “core” whose matrix comprises one or more monosaccharide(s), oligosaccharide(s), polysaccharide(s) or a combination thereof and at least one “layer” whose matrix comprises one or more metal oxide precursor(s), metal oxide(s) or metalloid compounds.

Multi-layer hybrid particles have the advantage of improving the thermal, chemical and mechanical stability of hybrid saccharide particles such as nanoparticles or microparticles, while remaining biosoluble.

The methods of the present invention for obtaining the particles are simple, economical and easy to implement. These methods allow the obtaining of biocompatible and biosoluble particles with the possibility of controlling their functionality. These methods also allows for example the diameter of the particles obtained to be finely modulated in a controlled manner by varying either the ratio of metal oxide precursor(s), metal oxide(s) or metalloid(s):monomer or organic polymer:catalyst, or the ratio of metal, metal oxide or metalloid(s):monomer or organic polymer:saturated carbohydrate solution or the reaction volume.

The non-hybrid multi-enveloped particles of the present invention, allow a delayed or prolonged release of a biologically active molecule. Preferably, for such a therapeutic application, the prolonged release can be modulated by superimposing layers comprising different monosaccharide(s), oligosaccharide(s) and/or polysaccharide(s), in particular by superimposing layers comprising oligosaccharide(s) which are longer and longer and whose solubilization/consumption kinetics are therefore slower and slower.

The polymers such as gels of the present invention, e.g., sol-gels are for example obtained by the methods of the present invention for the preparation of the polymer. The gels are preferably prepared by using an acid catalyst (such as HNO₃). The reaction volume is for example reduced or diluted, e.g., with water, or a gelling agent is added, in order to achieve the desired gel consistency. Preferably, the pH of the gel is kept below 1.5 to guarantee the stability of the gel and to reduce the gelling time. The main advantage of gels lies in the easy preparation of thin sheets with a very high homogeneity and a very wide choice of mixes including different types of particles (hybrid/non hybrid, mixed/non mixed), of different diameters and compositions.

The sheets are for example obtained from a stable, clear sol-gel solution or a mixture of two or more sol-gels without emulsions prior to mixing. Such sheets form for example a film of the present invention. The mixture comprises a volume ratio of the different constituents, a ratio appropriate to achieve the final physicochemical, photoluminescent, magnetic or electronic properties that the film must possess. The solution or mixture of solutions is then heated. This heating increases the viscosity of the solution or mixture of solutions to a certain value and allows, in the case of a mixture of sol-gel solutions, a good homogenization. The temperature and heating time are chosen according to the constituents of the gel and according to the biologically active molecule(s) optionally contained in the gel.

The sheets are for example obtained by any suitable means and any suitable methods allowing the deposition of the sol-gel solution, or mixture of solutions, on a suitable substrate. Preferably, this is done by immersion (dip-coating), spin-coating or evaporation under low pressure.

Polymers, gels or particles of the present invention are for example prepared by conventional sol-gel synthesis or any of its modifications known in the art. The particles are biocompatible, produced for example at low temperature and easily amenable to large scale production. They are less expensive to manufacture.

The sol-gel process comprises for example the following steps: preparation of a solution or suspension, of a precursor formed by a compound of the element (M) such as silica forming the oxide or alkoxide; hydrolysis (acid or base catalyzed) of the precursor to form M—OH groups. The so obtained mixture, i.e. a solution or a colloidal suspension, is named sol; polycondensation of the M—OH or M—OR groups according to the reactions

M—OH+M—OH->M—O—M+H₂O and M—OR+M—OH—>M—O—M+ROH

characterized by an increase of the liquid viscosity (gelation) and by the contemporaneous formation of a matrix called gel. The gel may be dried to a porous monolithic body or dried by a controlled solvent-evaporation, to produce xerogels, or by a solvent supercritical extraction to produce aerogels.

Silica and silicium dioxide, respectively, are used in medicine for example in form of Nitrostat®, which is already on the market. Nitrostat® is a stabilized sublingual compressed nitroglycerin tablet that contains 0.3 mg, 0.4 mg, or 0.6 mg nitroglycerin; as well as lactose monohydrate, NF; glyceryl monostearate, NF; pregelatinized starch, NF; calcium stearate, NF powder; and silicium dioxide, colloidal, NF. Such medical silica or silicium dioxide is for example part of the particle of the present invention.

If the polymer, gel, particle, composition or film of the present invention loaded with an active agent such as insulin is sublingually administered, the active agent crosses the sublingual mucosa. Insulin for example reduces the blood glucose level in a diabetic subject model such as a domestic pig and Göttingen minipig, respectively, to a physiological level and maintains a steady state for several hours for example up to 6 h such as 4 to 6 h.

The invention is not limited to the particular steps and materials disclosed herein. The terminology used herein is used for the purpose of describing particular embodiments only and is not intended to be limiting as the scope of the present invention will be limited only by the appended claims and equivalents thereof.

### EXAMPLES

The present invention is further illustrated by the following examples and figures without limiting the invention to the examples.

### Example 1: Preparation of Non-Hybrid Saccharide Particles

A saccharide-saturated alcoholic solution is prepared by mixing a large excess of saccharide (10 to 40 g or 20 g) in a 20%, 40% or 60% hydro-alcoholic solution under magnetic stirring and at room temperature for 48 hours. The alcohol of the hydro-alcoholic solution is methanol, ethanol, propanol, butanol, isopropyl alcohol, isobutyl alcohol or a mixture thereof. A polycondensation catalyst solution is prepared by adding 0.1-3 mL (e.g., 0.1, 1, 1.5, 2, 2.5 or 3 mL) of an aqueous solution of ammonium hydroxide (10%, 15%, 20%, 25%, 28% or 30%) to 15 mL of absolute alcohol such as methanol, ethanol, propanol, butanol, isopropyl alcohol, isobutyl alcohol or a mixture thereof. After homogenization under magnetic stirring for 15 minutes, 1 mL of the filtered hydro-alcoholic solution saturated with saccharide such as glucose, fructose, maltose, sucrose or a mixture thereof is added dropwise to the catalyst solution under magnetic stirring and at room temperature. After about two hours of stirring, the reaction medium becomes turbid and small whitish particles are formed. Agitation is continued for a further 24 or 48 hours. The nanoparticles are collected by centrifugation, washed with anhydrous absolute alcohol such as methanol, ethanol, propanol, butanol, isopropyl alcohol, isobutyl alcohol or a mixture thereof (3 times, 3 mL or 5 mL) and optionally dried in an oven.

### Example 2: Preparation of Hybrid Silica/Saccharide Single Layer Particles

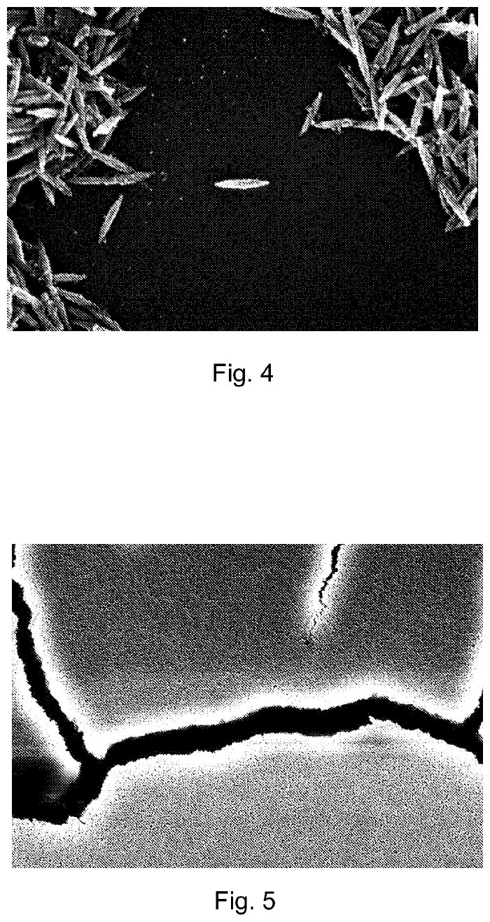

A saccharide-saturated hydro-alcoholic solution with 20%, 40% or 60% alcohol is prepared according to example 1. A catalyst solution is prepared by adding 1 mL of an aqueous solution of ammonium hydroxide (10%, 15%, 20%, 25%, 28% or 30%) to 15 mL of absolute alcohol such as methanol, ethanol, propanol, butanol, isopropyl alcohol, isobutyl alcohol or a mixture thereof under magnetic agitation for 15 minutes. 200 μL of TEOS or TMOS, are premixed thoroughly with 400 μL of the saccharide-saturated, hydro-alcoholic solution. Then 600 μL of this mixture is added dropwise to the catalyst solution under magnetic stirring at room temperature. Agitation is continued for 24 or 48 hours. The nanoparticles are collected by centrifugation, then washed with methanol, then with water and optionally dried in an oven (FIG. 3).

### Example 3: Preparation of Silica/Saccharide Rice-Like Nanoparticles

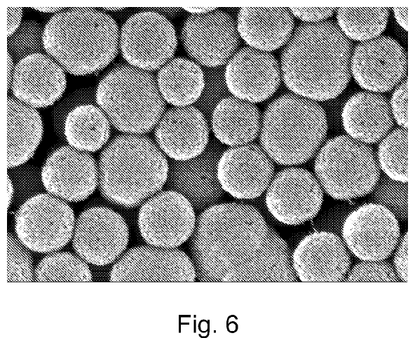

A saccharide-saturated hydro-alcoholic solution with 40% alcohol is prepared according to example 1. A solution is prepared by dissolving the surfactant cetyltrimethylammonium bromide (CTAB) in deionized water and its pH adjusted to 9.6 with ammonium hydroxide. To this solution 1-hexanol is added in a molar ratio of CTAB:1-hexanol being 1:1. Afterwards 200 μL of TEOS is premixed thoroughly with 400 μL of the saccharide-saturated solution and added dropwise to the mixture of CATB:1-hexanol. The reaction mixture with a molar ratio CTAB:TEOS:NH₃:H₂O being 0.11:1:10:525 is then allowed to react at 800 under continuous stirring for 10 hours. The nanorice hybrid particles (FIG. 4) were collected by centrifugation followed by washing and drying.

### Example 4: Preparation of Titanium/Saccharide Hybrid Particles with a Single Layer

A saccharide-saturated hydro-alcoholic solution is prepared according to the method as described in Example 1. 10 mL of this solution is premixed with 5 mL titanium isopropoxide (Ti[OCH(CH₃)₂]₄) and 10 mL isopropanol. To this solution under stirring, 200 mL of an HNOs nitric acid solution (0.01M concentration) (polycondensation catalyst) preheated to 80° C. under vigorous magnetic stirring is added. The rapid hydrolysis of the titanium (IV) isopropoxide leads to a whitish coloring of the solution. The reaction mixture is heated under reflux for 48 hours. 5 mL of the carbohydrate-saturated solution is added to the mixture and the reaction medium is cooled to room temperature. The nanoparticles are collected by filtration and then washed intensively with isopropanol (3 times/3 mL), water (3 times/3 mL) and dried in an oven.

### Example 5: Preparation of Silica/Saccharide Hybrid Particles with a Double Layer

Non-hybrid saccharide particles are prepared by the method as described in Example 1, but are not isolated. To the reaction medium, 0.2 ml of an aqueous solution of ammonium hydroxide (28%) and then 1 ml of TEOS are added dropwise at room temperature with stirring. The reaction is continued under stirring for 24 hours. The nanoparticles are collected by centrifugation, then washed with absolute alcohol (3 times, 3 mL), then dried in an oven.

### Example 6: Alternative Preparation of Silica/Saccharide Hybrid Particles with a Double Layer

The particles are prepared according to the method as described in Example 5 except that the TEOS is replaced by a TEOS/hydroalcoholic saccharide-saturated solution mixture in a ratio of 1:1 (v/v), the saccharide-saturated hydro-alcoholic solution being prepared according to Example 1.

### Example 7: Preparation of Single Layer Gold/Saccharide Hybrid Particles

5 mL of a saccharide-saturated 40% alcohol solution, prepared according to Example 1, is mixed with 10 mL of a solution of tetrachlorauric acid (HAuCl₄; 1 mM) with magneti stirring at room temperature. 600 μL of a freshly prepared cold solution of NaBH₄ is added rapidly with vigorous stirring. The color of the solution rapidly changes from yellow to a purple-brown indicating the formation of colloidal gold.

### Example 8: Alternative for the Preparation Of Single Layer Gold/Saccharide Hybrid Particles

A 40% saccharide-saturated hydro-alcoholic solution is prepared according to Example 1. 10 mL of this solution is mixed with 10 mL of an aqueous solution of HAuCL₄ 3H₂O (5 mM). The mixture is heated to boiling. Then 10 mL of a 0.5% sodium citrate dihydrate solution (HOC(COONa)(CH₂COONa)₂ 2H₂O) is added with magnetic stirring and heating is continued until a red color characteristic of colloidal gold formation is obtained.

### Example 9: Preparation of Vectors Comprising Plasmid DNA

A saccharide-saturated 40% hydro-alcoholic solution is prepared according to the method as described in Example 1. A polycondensation catalyst solution is prepared by adding 200 μL of an aqueous ammonium hydroxide solution (28%) to 10 mL of absolute ethanol with magnetic stirring. 600 μL of the saccharide-saturated hydroalcoholic solution is pre-mixed with 200 μL of a hydro-alcoholic plasmid DNA solution (5 μg/μL) and then added dropwise to the catalyst solution with stirring at room temperature. Agitation is continued until small whitish particles appear. At this stage, and without the particles being isolated, 60 μL of an aqueous ammonium hydroxide solution (28%) is added to the mixture and stirred for 5 minutes. Then 1.5 μL of 3-aminopropyl-trimethoxysilane (APTMES) and 125 μL of TEOS were premixed and added drop by drop to the mixture at room temperature. The reaction is continued under stirring for 12 hours. The nanoparticles are collected by centrifugation, then washed with absolute ethanol (3 times, 3 mL) and dried under vacuum and at low temperature.

### Example 10: Alternative Preparation of Vectors Comprising Plasmid DNA

The particles are prepared by the method as described in Example 9 except that 3-aminopropyl-trimethoxysilane (APTMES) is replaced by 3-aminopropyl-triethoxysilane (APTES), 3-glycidyloxypropyl-trimethoxysilane, 3-mercaptopropyl-trimethoxysilane or 3-mercaptopropyl-triethoxysilane.

### Example 11: Preparation of Vectors Comprising an Enzyme

The nanoparticles are prepared according to Example 9 with the exception that plasmid DNA solution is replaced by a hydro-alcoholic solution of the enzyme “Alkaline Laccase Ssl1” (Streptomyces sviceus) at 2 μg/μL and 3-aminopropyl-trimethoxysilane (APTMES) is replaced by 3-mercaptopropyl-triethoxysilane.

### Example 12: Preparation of Single Layer Particles Comprising Insulin (Type-A)

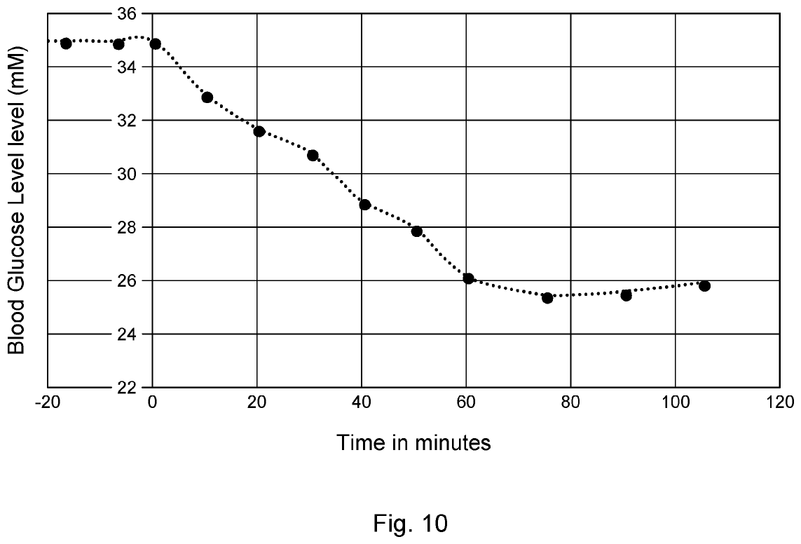

A saccharide-saturated 40% hydro-alcoholic solution is prepared according to the method as described in Example 1. 200 μL of the saccharide-saturated hydroalcoholic solution is pre-mixed with 1 mL of a hydro-alcoholic solution of Human insulin (5 mg/mL) and then added dropwise to an alcoholic solution under vigorous stirring for 2 hours. A white precipitate forms. To this solution, 100 μl of a polycondensation catalyst solution is added followed by a premixed alcoholic solution of 0.5 mL of the saccharide-saturated solution and 0.5 mL of TEOS. The reaction is continued under stirring for 12 hours. The insulin nanoparticles are collected by centrifugation, then washed with absolute ethanol (3 times, 3 mL) and dried under vacuum and at low temperature via lyophilization (FIG. 8).

### Example 13: Preparation of Multi-Layer Particles Comprising Insulin (AF-Type)

The nanoparticles are prepared according to Example 12 for obtaining a single layer particle comprising insulin, but not isolated. Before collecting the nanoparticles by centrifugation, two steps are added:
(a) 1 mL of a hydro-alcoholic solution of recombinant human insulin (5 mg/mL) is added dropwise and the mixture is further stirred 30 min.
(b) afterwards a premixed alcoholic solution of 0.5 mL of the saccharide-saturated solution and 0.5 mL of TEOS is added to build-up the second shell around insulin. The mixture is stirred for additional 10 or 12 hours.

Steps a) and b) are repeated one or more time (up to 5 times) for obtaining a multi-layer particle comprising insulin.

### Example 14: Preparation of Porous Particles Loaded with Insulin (BF-Type)

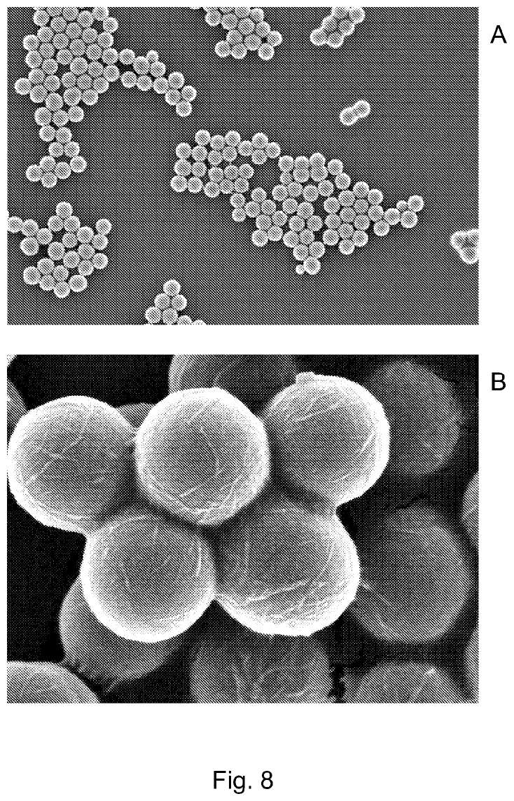

Silica-saccharide particles are prepared by the method as described in Example 2. After the particles are collected by centrifugation, they are washed thoroughly with hot water (3 times, 3 mL), then with hot ethanol (3 times, 3 mL), to remove all unreacted saccharide molecules, and dried in an oven or lyophilized. The so-obtained particles present large pores at the surface but also in the bulk, because the wash-out of unattached saccharose leaves empty holes (FIG. 6). After the drying step, the particles are immersed in a hydro-alcoholic solution of Human insulin (10 mg/mL) and shacked gently for 24 hours at 8° C. The insulin loaded particles are collected by centrifugation, and dried under vacuum at low temperature.

### Example 15: Preparation of Porous Particles Loaded with Leptin (A Glucagon Suppressor)

Silica-saccharide particles are prepared by the method as described in Example 14, with the exception that human insulin solution is replaced by a hydro-alcoholic solution of Leptin at 800 μg/mL, optionally in the presence of 3-aminopropyl-trimethoxysilane (APTMES) at 100 μg/mL. Alternatively, similar porous particles can be loaded with a mixture of insulin and leptin, wherein the hydro-alcoholic solution is prepared by dissolving 5 mg of human insulin and 1 mg of leptin in 1 ml water-ethanol (1:1). The mixed solution is applied to the porous particles and the mixture is gently shacked for 24 to 48 hrs at 4C.

### Example 16: Preparation of a Gel Comprising Non-Hybrid and Non-Mixed Particles

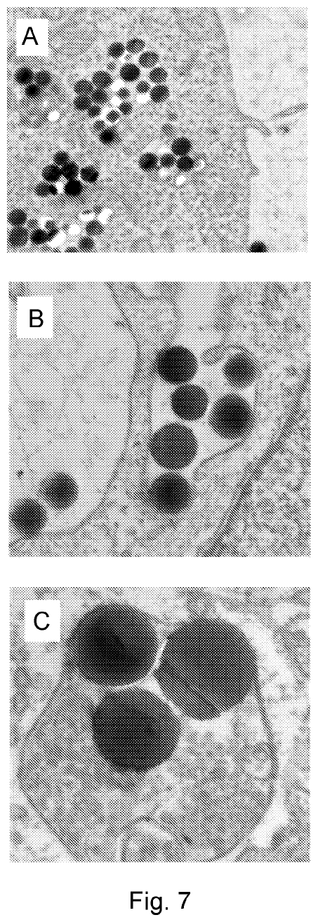

Particles are produced according to Example 1 in reduced volume, without being collected. The volume is reduced or diluted to the desired gel consistency (FIG. 5).

### Example 17: Preparation of a Film Comprising Non-Hybrid and Non-Mixed Particles

The gel obtained in Example 16 is applied to a glass plate previously cleaned with a solution of chromic acid, a detergent solution and distilled water. The cleaned plate is immersed for a few seconds (1 to 2 seconds) in a beaker containing the gel from example 16 at a concentration of 0.1M/L and a pH≈8. The glass plates are then immersed in a hot water solution at a temperature between 90 and 95° C. The immersion process is repeated several times until the chosen thickness is achieved. The resulting films are dried in an oven at 150° C.

### Example 18: Biodegradability Tests of the Nanoparticles

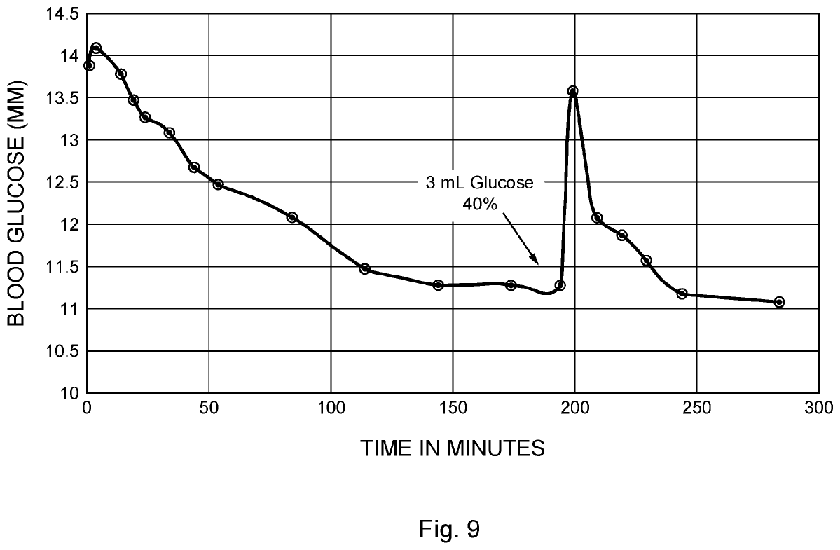

MDCKII Cells Madin Darby Canine Cells were grown in DMEM-F12 media with 10% PBS in 6-well plates (area=9.5 cm2) to obtain a sufficient cell number to prepare a 1-mm3 pellet (˜106 cells or more). Culture Media contained 1% penicillin-streptomycin as an antibiotic. The cells were maintained in a 5% CO₂ incubator at 37° C. and 100% humidity. Cells are typically grown to at least 70% confluency before testing. Silica-saccharide nanoparticles were weighed in a dry powder form on an analytical mass balance, then suspended in deionized water at a concentration of 1 mg/ml and retained as stock solutions. Stock NP solutions (1 mg/ml) are diluted into cell-culture media to working solutions with concentrations of 100 μg/ml. Sonicate working solutions for 30 s at 35-40 W for better dispersion and added to culture media. After 5 hours of incubation the growth media was aspired from the cells and replaced by fresh media. Afterwards the cells were transferred to the incubator and maintained at 37° C. and 5% CO₂ for 4 days. Fixation solution: Fresh fixative solution was prepared by combining glutaraldehyde and formaldehyde in PBS at final concentrations of 2.5% each and used immediately. Osmium tetroxide: dilute the osmium tetroxide in PBS to 1% as a post-fixative (equal parts PBS and 2% osmium tetroxide) (FIG. 7).

### Protocol for the Preparation of TEM Samples

Fixation of the cells: After appropriate incubation time (24 hrs, 48 hrs and 72 hrs), the cells were rinsed thoroughly from the NP solution with fresh dosing media (containing no serum) at room temperature 23 times for at least 5 min each time. Aspire the media after the last wash. The cells are detached from the well plate using trypsin, a pipette or a scrape and then pipetted into a conic shaped Eppendorf. Centrifugation of the cells for 5 min at 1,000 g at room temperature allows a pellet of 0.5 to 1 mm to form at the bottom of the Eppendorf. To this pellet ˜1 mL of fresh 2.5% glutaraldehyde/formaldehyde in PBS at room temperature for 2 hrs. After the fixation is complete, rinse thoroughly the pellet with PBS, three times for 10 min each. Add ˜1 ml of 1% osmium tetroxide in PBS to the cell pellet for 1 h. Rinse the pellet with PBS five times for 10 min each and then with double-distilled water (ddH2O) two times for 10 min each. Remove ddH₂O and start dehydration through a graded series of ethanol concentrations (50, 70, 90 and 100%) for 5-15 min each, for replacing the water in the sample with ethanol.

Resin embedding and curing of the cell pellet: Add a 50:50 resin:ethanol mixture for 30-45 min or longer while taking care to avoid the formation of bubbles during resin mixing by stirring slowly. Replace the diluted resin mixture with 100% resin. Allow resin to infiltrate into the sample and cure overnight (˜15 h). If the sample appears to be soft or tacky, continue curing before trimming or sectioning.

Trimming of sample: Prepare the sample block face after the cells have been embedded and cured. Use an ultramicrotome to cut very thin sections of the cells pellet using a diamond knife. The recommended section thickness for embedded cells is between 50-100 nm. Produce a sufficient number of sections floating on the water surface and collect them onto a TEM grid (300-mesh Cu, with support film). Allow sections on grids to dry for a few minutes, then carefully place grids in a grid storage box using fine-tipped tweezers.

### Example 19: In-Vivo Efficacy Studies of Insulin Encapsulated Hybrid Nanoparticles in Domestic Pigs and Göttingen Minipigs

### Project Type: Chronic, In Vivo Large Animal Experiments

Testing the sublingual application of SLIM particles loaded with insulin in normoglycemic and STZ diabetic Göttingen domestic pigs and Göttingen minipigs in a chronic in vivo study.

The purpose of this chronic, large animal in vivo study was to test the effective penetration of insulin containing particles through the sublingual mucosa, compare the effect with the subcutaneously applied human recombinant, measure the blood glucose changes and analyses the plasma insulin levels. Domestic pigs were also included in the study to compare the glucose lowering effect of insulin-loaded particles in the Göttingen minipigs and domestic pigs. The test compounds were applied s.l. and s.c. to the pigs being under the anesthetic propofol (Amnesia).

### Regulatory Guidelines on Animal Health and Welfare:

The study was designed in accordance with accepted pharmacological principles in order to meet the requirements of the principles of Hungarian Act 1998: XXVlll regulating animal protection (latest modified by Act 2011 CLVIII) and in Government Decree 40/2013 on animal experiments.

EEC Directive 2004/27/EC of the European Parliament and of the Council of Mar. 31, 2004 amending Directive 2001/83/EC on the Community code relating to medical products for human use (Official Journal L-136, 30/04/2004, pp. 34-57).

Handling and care of the animals are conducted according to the Guide for the Care and Use of Laboratory Animals, NRC, 2011 and Directive 2010/63/EU (European Parliament and Council, took full effect on 1, Jan. 2013).

Special permission for animal studies under number PE/EA/1026-8/2019 was issued from Pest County Government Office of Food Safety and Animal Health Directorate.

### Rationale of the Experiments

Pigs are good models of human diabetes and suitable for testing insulin replacement therapy. The pigs are omnivores just like the humans. The glucose metabolism in pigs is also similar to the human carbohydrate metabolism regulation. The regulation of insulin release and the pharmacokinetics of insulin are also similar. In the present study the aim was to test s.l. application of different insulin containing microcapsules. In this regard, the pig represents also the best animal model. The mucosa of the pigs mouth is very similar to the human mucosa and a large part of the porcine mucosa is a non-corneal epithelium, which provides good absorption, thus suitable for testing sublingual and/or transbuccal modalities.

The common laboratory animals, as rodents, have only a limited non-corneal area of the buccal mucosa, where the border between corneal and non-corneal epithelium can be hardly delineated, so the correct testing is not possible.

In this study, we first recorded the effect of s.c. application of human recombinant insulin as reference compounds and s.c. and s.l. applications of micro-compounds. The tests were conducted in normoglycemic state then the insulin-producing pancreatic islets beta cells were destroyed by i.v. application of streptozotocin (STZ) in order to induce diabetes. The glucose levels of the pigs were followed and monitored for at least 3 days. The application of different micro formulations started only after stabilization of the hyperglycemic blood glucose level.

In the study 4 Göttingen mini-pigs and 4 domestic pigs were involved.

### Study Design, and Experimental Protocol

In the study 2 different series were conducted:
in 4 domestic pigs, the SLIM particles were tested in normoglycemic and in STZ evoked diabetic stage (one domestic pig served as normal control without any treatment),
in 4 Göttingen mini-pigs the SLIM particles were tested in normoglycemic and in STZ evoked diabetic stage (one mini-pig served as normal control without any treatment).

### The Test System

TABLE 3
Experimental Animals

| | |
|---|---|
| Species and strain | Göttingen minipigs |
| Source | Ellegaard Gottingen Minipigs A/S, Dalmose |
| | Denmark |
| Hygienic level at | SPF |
| arrival | |
| Number of animals | 4 |
| Sex | 4 female |
| Age of animals | 16 weeks at the start of acclimatization |
| Body weight of | 23-24 kg |
| animals | |
| Acclimatization | 1 week before the start of testing |
| Species and strain | Domestic pig (Sus scrofa domesticus), mixed breed |
| | of Duroc and Hungarian landrace |
| Source | Research Institute of Animal Breeding and |
| | Nutrition, Herceghalom, Hungary |
| Hygienic level at | non-SPF |
| arrival | |
| Number of animals | 4 |
| Sex | 2 male and 2 female |
| Age of animals | 16 weeks at the start of acclimatization |
| Body weight of | 21-23 kg |
| animals | |
| Acclimatization | 1 week before the start of testing |
| Vaccination | 1 mL Noromectin inj. at 70 days after birth* |
| | 1 ml Ferroferon inj. within 72 hours after birth** |
| | 1 ml Ingelvac-Cirkoflex inj. at 32-35 days after |
| | birth*** |

*For protection against external and internal parasites.
**For prophylaxis of iron deficiency anemia in piglets.
**For protection against PCVAD—Porcine Circovirus Associated Disease.

Domestic pigs ear notching done soon after birth were used in the study. Notching provide a permanent identification system with information about the pig parentage, birth weight, medication etc.

TABLE 4
Husbandry, Food and Water Supply

| | |
|---|---|
| Husbandry: | individual caging, but visual and smell contact. During |
| | the cleaning socialization is possible. |
| Cage type: | steel with concrete floor and straw |
| Cage size: | 2 m × 3 m |
| Diet: | normal starter food for domestic pigs (composition, |
| | certificate see in the appendix) |
| Diet format: | Granulate |
| Availability: | restricted, offered once daily, in certain occasions |
| | during the diabetic state two times daily |
| Water supply: | tap water |
| Availability: | ad libitum, but restricted after the sublingual treatment |

The drinking water was periodically analyzed and was considered not to contain any contaminants that could affect the health of the animals and consequently the integrity of the study.

### Animal Identification:

Each animal was uniquely identified via ear tags.

### Description of the Test Procedure

### 1. Acclimatization, Chronic Catheter Installation

On the first day of acclimatization a central venous catheter was installed (Certofix®Mono B-Braun V430) for each domestic pig and Göttingen minipig. The pigs were fasted overnight before the day of operation, but supplied with water ad libitum. The animals were pre-anesthetized intramuscularly with Calypsol/Xilazine (2/1.8 mL, based on body weight) injections in the stalls to avoid stress, and were transported to the operating room. The anesthesia was maintained using isoflurane inhalation narcosis (2-2.5%) with oxygen. through the right external jugular vein. The venous catheters were introduced by aseptic technique into the left external jugular vein. The end of the catheter was pulled subcutaneously to the back part of the head between the ears, and fixed by sutures, in two layers. The catheter served for blood sampling for insulin and glucose measurements. All surgical points were disinfected by liberal application of polyvidone iodide, and the free end of the catheter was fixed on the head of the pig. To avoid a potential infection Betamox (amoxicillin, 15 mg/kg dose) was injected i.m. and for pain relief Rheumocam (0.6 mg/kg) was also given (i.m.).

During the acclimatization week the pigs were handled daily to become familiar with the operator, and acquainted to the blood sampling from the chronic catheter without stress.

### Testing the Short-Term Anesthesia Methods

For sublingual (s.l.) treatment it is necessary to anesthetize the animals as to allow the application of the insulin complexes onto the sublingual mucosa, but long-term anesthesia is undesirable as it may affect insulin/glucose metabolism. For this purpose Propofol (Amnesia, 5 mg/kg, 20 mg/mL) was injected i.v. and 30 seconds later the pig was laying down and the jaw was relaxed after 60 seconds. After 10 minutes the animal started to move, and 2×1 mL additional propofol dose was injected i.v. With this dosing, the animal was sleeping for 20 to 23 minutes, the test material was applied sublingually 5 min after the Amnesia administration. The first eye movement occurred after 23 minutes, and the pig was standing up on four legs after an additional minute quietly without any excitement. 30 minutes after the injection of anesthesia the pig started walking and drinking. Blood glucose measurements were taken at 20 minutes before the anesthesia, then 5, 10, 15 and 30 minutes after and were in the normal range.

### 2. Generating an Insulin Deficient Diabetic State of the Pigs

The insulin deficient state was produced by chemical destruction of the Langerhans islets' beta cells of the pancreas responsible for insulin secretion. The Streptozocin (STZ; 2-deoxy-2-(3-(methyl-3-nitrosoureido)-D-glucopyranose)) is a known chemical compound that selectively destroys the beta cells and lead to a good type I insulin dependent diabetes model (IDDM). For pigs, the recommended STZ dose is 150 mg/kg i.v. Such a dose of STZ after a couple of hours' results in low insulin and high blood glucose levels. After several fluctuations, the insulin and BG levels start to stabilize after 2 to 3 days.

Mechanism of STZ action: STZ enters the pancreatic beta cells through the GLUT-2 glucose transporter and exerts its effects at several attack points:
1—it causes DNA alkylation of pancreatic beta cells;
2—STZ as a donor of NO also contributes to the damage to the DNA of beta cells;
3—it generates reactive oxygen species;
4—it generates mitochondrial damage by generating superoxide anions, hydrogen peroxide and hydroxyl radicals by enhancing xanthine oxidase activity;
5—STZ also inhibits the Krebs cycle and significantly reduces oxygen consumption;
6—it causes loss of ATP production and faster ATP degradation.

All these effects lead to a rapid and drastic reduction in insulin production.

The so-created insulin deficient diabetic pig model for carbohydrate, fat and amino acid metabolism with regard to non-treated human lDDM often used, and it is a good model. It is suitable for the insulin supplement treatment, testing of insulin replacement by various methods and assessing the glucose/insulin kinetics.

The application is recommended before feeding and after blood glucose and body weight measurement, 150 mg/kg of freshly prepared streptozocin solution was injected, under propofol (Amnesia 3 mL) narcosis, according to the body weight in the pigs.

STZ was purchased from Sigma-Aldrich Co. (ref: S0130), it was dissolved in 100 mmol/L disodium citrate buffer solution, pH 4.5, at a concentration of 50 mg/mL, and administered through the central venous catheter by slow i.v. injection (approximately within 2 min.).

The glycemic status was controlled and monitored subsequently by repeated BG measurements during the following days and corrections were applied when necessary (i.e. insulin was administered in case of high glucose level (>30 mM/L), or glucose injection through the central venous catheter) if necessary (blood glucose <2 mM/L).

The BG level monitoring was followed for 3-4 days, until a stable STZ diabetic stage was confirmed.

### 3. Testing the Insulin Containing SLIM Formulations in STZ Diabetic Domestic Pigs and Mini-Pigs, Collecting Tissue Samples

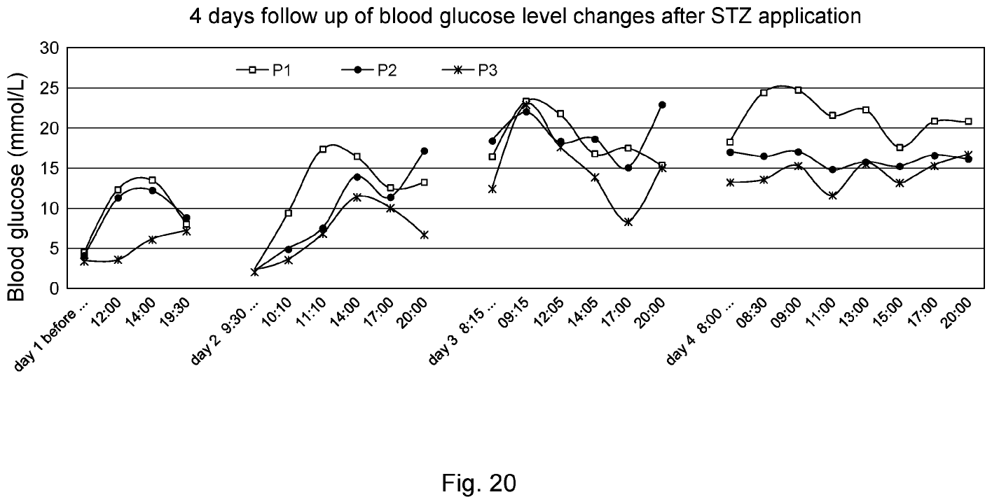

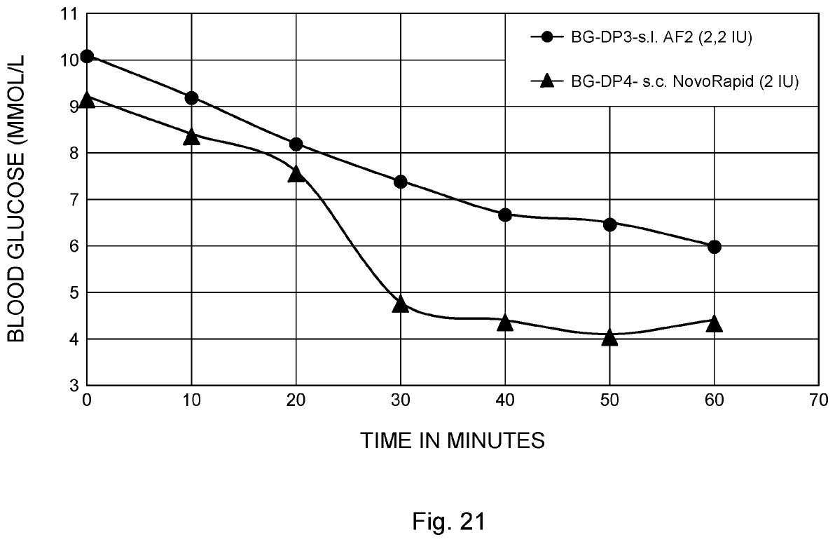

After stabilizing the STZ diabetic state in domestic-pigs (FIG. 19) and mini-pigs (FIG. 20), started the testing with the reference substances human insulin by subcutaneous application and the SLIM insulin containing formulations by subcutaneous (s.c.) and sublingual (s.l.) application. The test and reference compounds were applied mostly under the tongue, but in few cases the s.c. treatments also were applied in non-anesthetized animals (for comparison). The Anesia anesthetic state was maintained either during the full time of observation or only for a shorter period to make the s.l. treatment and thereafter ˜30 minutes and in the later phase the BG measurement and blood sampling for insulin determination occurred in the stall in awake condition from the central venous catheter.

On the last day of the study, by Euthasol and concentrated KCl i.v. injection the animals were sacrificed and tissue samples were collected for histology samples and for TEM embedding. From 13 organs have been collected samples (-Brain, -Liver, -Kidney, -Skeletal muscle, -Adipose tissue, -Stomach, -Heart, -Sublingual mucosa, -Lung, -Fat, -Spleen, -Pancreas, -intestine such as small intestine, -skin) immediately after sacrificing the pigs (on 14th of December, 2019 of the domestic pigs and on 15th the mini-pigs). One additional mini-pig was also sacrificed two days after arrival, without any treatment and one domestic pigs served also as a normal control, and from the same organs tissue samples were collected. The tissue samples were stored at 4° C. in paraformaldehyde (PFA, 10% in PBS) then paraffin-embedded for hematoxylin-eosin staining in the histology laboratory of 1st Pathology Institute of Semmelweis University. For TEM embedding ˜1×1 mm tissue samples were collected (5-7/tissue) and fixed overnight in 2.5% glutaraldehyde in 0.1 M sodium cacodylate buffer (pH 7.2), transported to the Anatomy Institute of Semmelweis University where 3-3 blocks were prepared from each organ.

### Example 20: In-Vivo Efficacy and Comparative Studies of Sublingual Application of SLIM Particles with Commercial Insulin (Novorapid, Novo Nordisk) in Normoglycemic Domestic Pigs

To compare the efficacy of SLIM particles with commercial insulin, normoglycemic domestic pigs were challenged by administering a low dose of insulin in two different ways. Domestic Pig 3 received SLIM-AF2 (AF, 2,2 IU) sublingually and domestic Pig 4 received commercial insulin (Novorapid, 2 IU) subcutaneously and the BG levels in both pigs were monitored and compared for 1 hour after the administration.

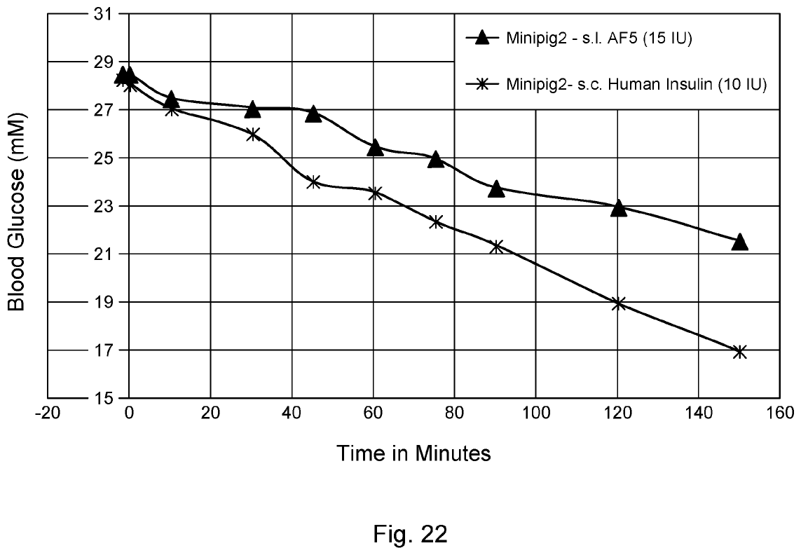

During the first 20 min following the application, the BG level of each animal starts to decline equally with relatively similar speed and kinetic (FIG. 21), showing that SLIM particles are readily absorbed through the mucosa followed by an immediate release of the encapsulated insulin in SLIM-AF2. In this first part of the graphs the two ways of administration are fairly comparable. After 20 min the BG level in Pig 4 starts to decline a bit faster before reaching a steady state at which the BG stabilizes for more than 20 min. Contrarily to this, the BG level of Pig 3 in this second part of the graph continue to decline linearly with almost the same kinetic over the whole period of 1 hour without reaching any steady state or a further increase. This result demonstrates the efficacy of the sublingual application of SLIM particles and suggests that the encapsulated insulin is active instantaneously after the application followed by a linear response to the sustained release of insulin from the particle over time.

Regulatory guidelines on animal health and welfare, rationale of the experiment, study Design, and experimental protocol, test system and husbandry, food and water supply according were followed according to Example 19.

### Example 21: In-Vivo Efficacy and Comparative Studies of Sublingual Application of SLIM Particles with Commercial Recombinant Human Insulin in Diabetic Göttingen Minipigs

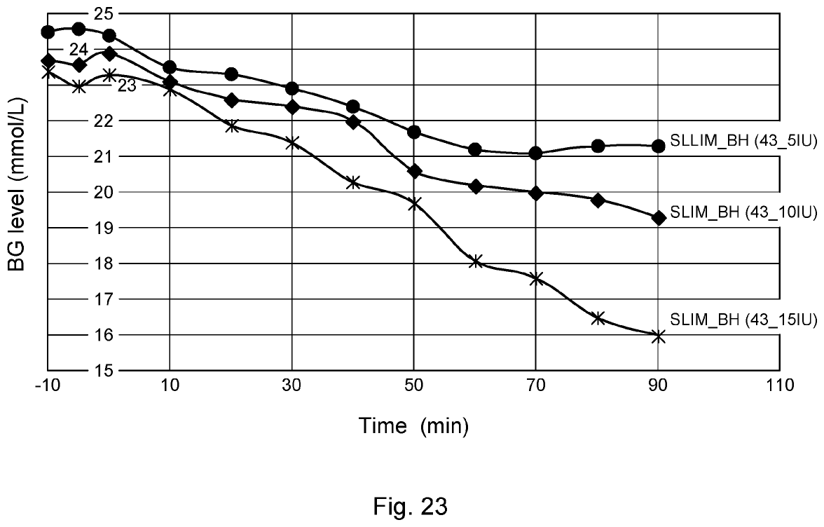

Corresponding to Example 20, the efficacy of SLIM particles was compared to subcutaneous injection of commercial recombinant human insulin in the same diabetic Göttingen minipig (Minipig 2) on different days. First day the animal received SLIM-AF5 (AF, 15 IU) particles sublingually and the second day was administered Recombinant Human Insulin (RHI, 10 IU) subcutaneously and the BG levels were monitored for 2H30 and compared (FIG. 22).

As depicted in FIG. 22, during the first 10 min the BG level starts immediately to decline after the application in both case with exactly similar speed and kinetic. However after this first phase and until the end of the BG's monitoring, the BG levels in the two graphs continue to decline linearly with the time but with a slightly different speed and slop. In the case of SLIM-AF5 it can be clearly shown that the speed of the BG decline is rather governed by the slow and sustained release of insulin from the sub-layers of the SLIM particles (AF-type) as compared to the free insulin supplied during the subcutaneous injection.

Regulatory guidelines on animal health and welfare, rationale of the experiment, study design, and experimental protocol, test system and husbandry, food and water supply according were followed according to Example 19.

### Example 22: In-Vivo Efficacy and Dose Effect Studies of Sublingual Application of SLIM Particles of BF-Type in Diabetic Domestic Pigs

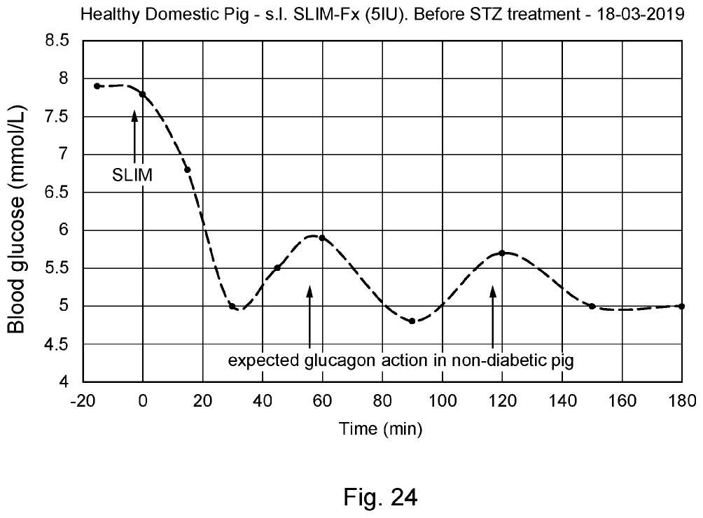

To study the dose effect of SLIM particles of BF-type on the kinetic of decrease of the BG level in diabetic domestic pigs, the SLIM particles were loaded with increasing doses of commercial Recombinant Human Insulin (5 IU, 10 IU and 15 IU) and were applied sublingually to the same diabetic domestic pig (Pig 2) on different days. The BG levels of the animal were monitored and compared for a period of 1H30 min after the application. As depicted in FIG. 23, the BG level of the domestic pig 2 declines, in each experiment, continuously and proportionally to the insulin dose loaded in the corresponding SLIM-BF particles. The sublingual application of SLIM-(BF, 15 IU) particles containing the higher dose of insulin produce a decline of the BG level that is faster and quicker than the effect produced by the SLIM-(BF, 10 IU), which produces itself a faster and quicker decrease of the BG level than SLIM-(BF-5 IU). This result suggests clearly that by adjusting the dose of insulin encapsulated in SLIM-BF particles (BF-type, conceived or fast release), it is possible to control and adjust the speed and the kinetic of decrease of the BG level in diabetic domestic pigs with a sublingual application.

### Example 23: In Vitro Assay for Assessment of Encapsulated Insulin in Preadipocytes

An in vitro assay has been designed to assess the bioactivity of an encapsulated compound such as insulin for example in preadipocytes. For example:
1. 3T3-L1 adipocytes cells are first incubated with fluorescently labelled 2-deoxyglucose (FITC-DG;
2. SLIM (Insulin encapsulated particles) are then added to the culture media containing FITC-DG);
3. Only active insulin can trigger the transport of glucose inside the cytoplasm through the activation of Glut4 Transporter;
4. Glucose metabolism was followed by confocal fluorescence microscopy.

### Example 24: Dose-Response of Porous SLIM Particles (Type B) on Blood Glucose Level in Healthy Pigs

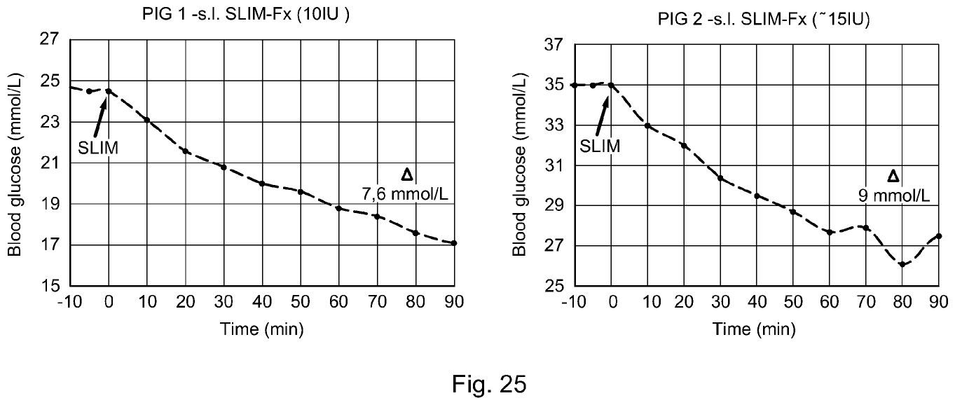

Multi-layer particles of the present invention (5 IU, see, e.g., Example 24) were sublingually administered to healthy pigs. Even in these pigs the blood glucose level decreased after administration of the inventive particles comprising insulin (see FIG. 24). This provides proof for high bioavailability of insulin in the particles of the present invention.

### Example 25: Dose-Pesponse of Porous SLIM Particles (Type B) on Blood Glucose Level in STZ-Diabetic Domestic Pigs

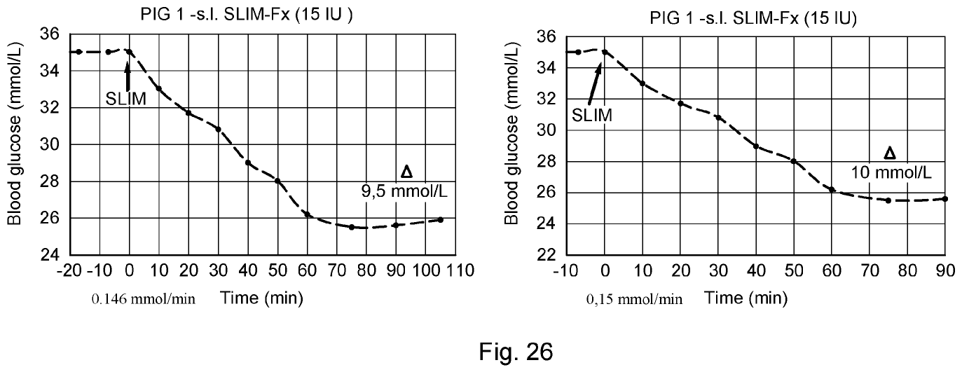

STZ-diabetic domestic pigs (pig 1 and pig 2) were independently treated with porous SLIM particles of the present invention comprising insulin. Administration of the particles was sublingual. The pigs received the same formulation of insulin comprising particles (Fx) at a dose of 10 IU and 15 IU, respectively. The experiments in two domestic pigs show the efficacy of the insulin comprising particles, the reproducibility and the dose-dependent response (see FIG. 25).

### Example 26: Reproducibility of the Dose-Response

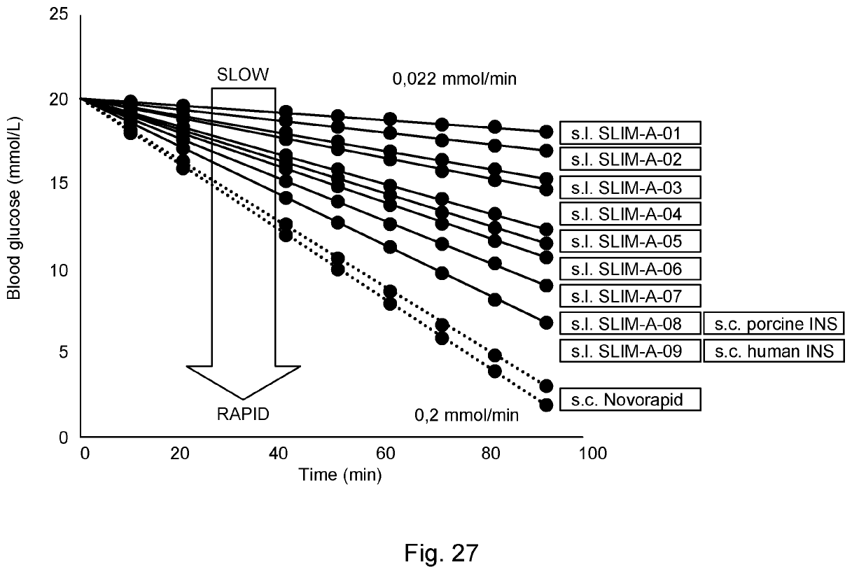

STZ-diabetic domestic pig 1 of Example 26 was treated with porous SLIM particles comprising insulin (15 IU) according to Example 25, but on a different day and the results on the blood glucose levels are highly comparable (see FIG. 26).

### Example 27: Pharmacokinetic Studies

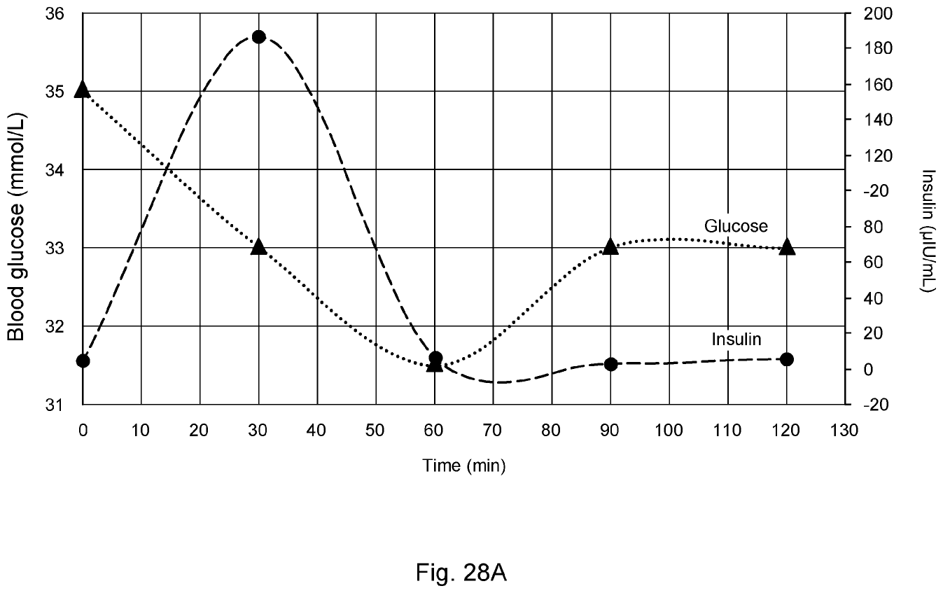

Multi-layer or porous SLIM particles comprising insulin in different amounts were sublingually administered to STZ-diabetic domestic pigs. The particles used in the experiments of FIG. 27 were:
SLIM-A-01=Multi-layer particles comprising human insulin (5 IU)
SLIM-A-02=Multi-layer particles comprising human insulin (10 IU)
SLIM-A-03=Multi-layer particles comprising human insulin (12 IU)
SLIM-A-04=Multi-layer particles comprising human insulin (15 IU)
SLIM-A-05=Porous particles comprising human insulin (10 IU)
SLIM-A-06=Porous particles comprising human insulin (12 IU)
SLIM-A-07=Porous particles comprising human insulin (15 IU)
SLIM-A-08=Mixed particles (multi-layer and porous) comprising human insulin (15 IU), and
SLIM-A-09=Mixed particles (multi-layer and porous) comprising human insulin (20 IU).

Insulin was released in the blood circulation at different speed, the kinetics were for example ranging from 0.022 mmol/min to 0.15 mmol/min. over 90 min. after sublingual administration. In comparison the subcutaneous administration of porcine or human insulin was tested for the effect on the blood glucose level (FIG. 27).

### Example 28: Monophasic Release Profile

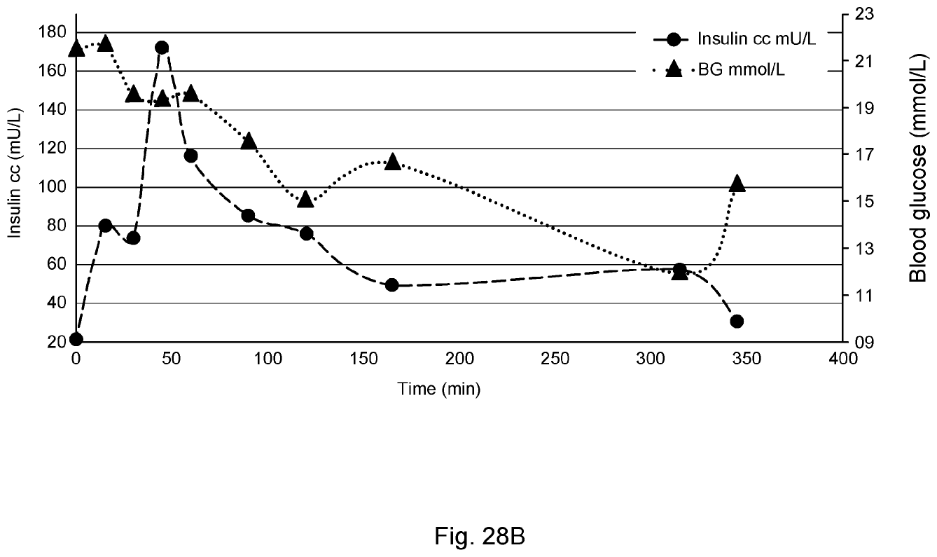

Multi-layer particles comprising 10 IU insulin (FIG. 28A) or 25 IU insulin (FIG. 28B) were administered to STZ-diabetic domestic pigs and STZ-diabetic minipigs, respectively. The glucose and insulin levels in the pigs were tested for 120 min. FIGS. 28A and 28B show a monophasic release profile of insulin in the blood of the pigs after sublingual application of the multi-layer SLIM particle.

### Example 29: Antibody Production

Two groups of mice were injected with particles of the present invention comprising different vaccines, i.e., antigens which were:
1) spike protein of SARS-COV2 (MW 135 kDa),
2) mixture of two peptides directed to the receptor binding domain (RBD) of the SARS-COV2 protein:
the first peptide comprises/consists of the receptor binding motif (part N-Nter) for SARS-COV2: GNYNYLYRLFRKSNLKPFERDISTEIYQAGSTPC (SEQ ID NO.1; 34aa, MW 4 kDa), and
the second peptide comprises/consists of the receptor binding motif (part C-Nter) for SARS-COV2: CYFPLQSYGFQPTNGVGYQPYR (SEQ ID NO.2; 22aa, MW 2.6 kDa).

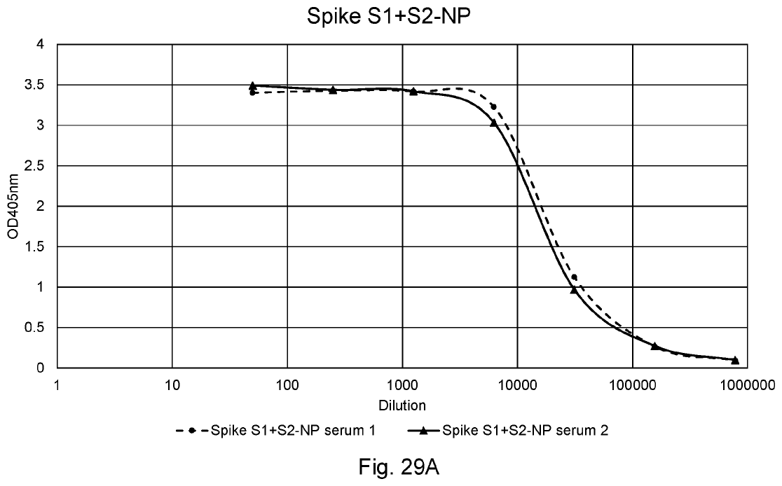

The experiments were performed in two replicates of mice for each antigen and the serum titles were 1/20,000 for the spike protein in all mice and 1/15,000 in average for the mixture of peptides comprising the receptor binding motif. FIG. 29A shows the serum title of the spike protein and FIG. 29B the serum title of the mixture of the two peptides comprising of the receptor binding motives.

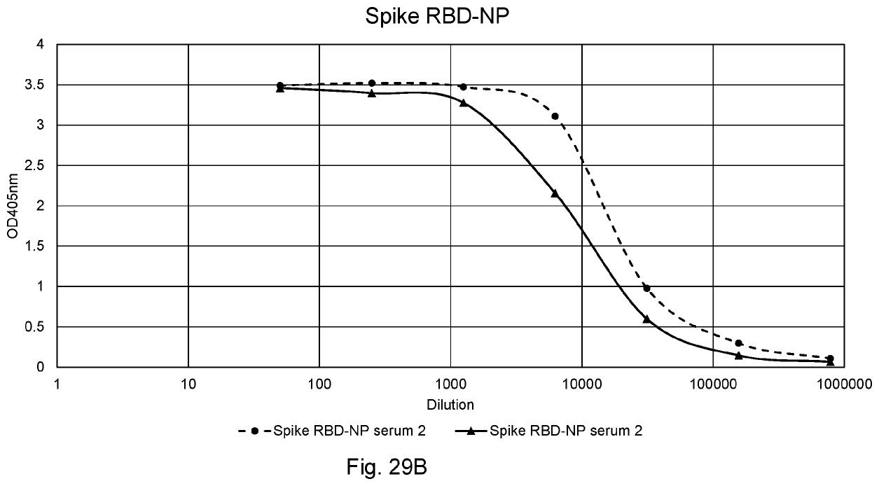

A control immunization based on KLH as protein carrier resulted in a serum title of 1/925 in average (FIG. 30).

Thus, the immunization with the present invention resulted in a 15 to 20-fold higher serum title.

### Example 30: Kinetic of Insulin Slow Release from SLIM Particles in In Vitro COS Cultured Cells or HEK273FT Cells Between 10 and 32 h

SLIM-AF [SL-30] particles comprising insulin in a concentration of 1.9 IU/mg or of 0.64 IU/mg were administered to COS 7 cells or HEK273FT cells to test the release of the insulin from the particles. The released insulin was determined with an ultrasensitive insulin ELISA (e.g., of Mercodia, Ref: 10-1132-01).

COS cells were cultured in a 24well plate and grown to 80% confluence. HEK273FT cells were cultured in a 24well plate and grown to 30% confluence. A stock solution of 10 mg/ml of SLIM-AF [SL-30] nanoparticles was prepared by dissolving the nanoparticles in DMEM+10%FCS. A working solutions of 15 ug/ml was prepared in the same medium.

The cell medium was removed from the COS 7 cells and replaced by medium containing the SLIM-AF [SL-30] nanoparticles (NP). The cells were incubated for 15 min and the NP containing medium was then removed and the cells were washed 2 times with fresh medium and incubated with fresh medium (DMEM+10% FCS) without NPs. Aliquots of 500 μl were then collected at 0 h, 0.5 h, 1 h, 2 h, 4 h, 6 h, 8 h, 10 h, 12 h, 14 h, 16 h, 18 h, 20 h, 24 h, 28 h, 32 h, centrifuged 2 min at 16000 g. The supernatants were analyzed using the ultrasensitive insulin ELISA: NPs with 1/100 dilution, kinetic samples with ½ dilution. The result is shown in FIG. 31.

To study the kinetic of insulin release from the SLIM-AF particles in HEK273FT cells, culture media were sampled from the well plate every hour (from T0 to T5H), centrifuged and stored at −20C until analysis. The results are shown in FIG. 32.

### Example 31: Kinetic of Insulin Fast Release from SLIM Particles Formulations in In Vitro Cultured HEK273FT Cells up to 5 h

Investigation of the kinetic of human insulin release from SLIM-AF particle compared to naked human insulin. The SLIM-particles comprising insulin in a concentration of 1.8 IU/mg were incubated with cultured HEK273FT cells in a 24-wells plate. The cultured HEK273FT cells were plated two days before the start of the test to reach 30% confluency. SLIM particle samples were suspended in appropriate volume of the culture media DMEM (10% FBS) and the calculated volumes were added to the plate.

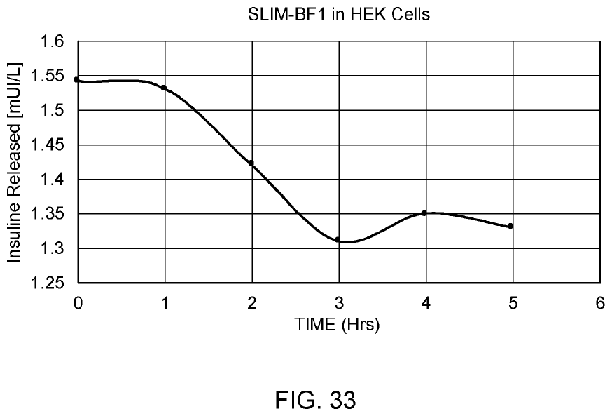

To study the kinetic of insulin release from the SLIM-BF particles, culture media were sampled from the well plate every hour (from T0 to T5H), centrifuged and stored at −20C until analysis. The results are shown in FIG. 33.

1. A method for the production of a polymer comprising the steps:
a) preparing a saturated solution of a carbon donor such as a carbohydrate, the carbon donor is dissolved in a water/alcohol solvent or water/alcohol/organic solvent wherein the ratio of water:alcohol is from 20:80 to 80:20 or wherein the ratio of water:alcohol:organic solvent is from 5:80:15 to 80:15:5,
b) mixing the saturated solution of step a) with a metal oxide precursor, a metal oxide or a combination thereof,
c) adding an alcoholic or hydro-alcoholic solution of a polycondensation catalyst to step a) and/or step b),
wherein all the steps are performed at a temperature in a range of about −20° C. to about 65° C., preferably about −5° C. to about 25° C. and the carbon donor and the metal oxide precursor, the metal oxide or a combination thereof form a gel,
or
a) preparing a saturated solution of a carbon donor such as a carbohydrate, the carbon donor is dissolved in a water/alcohol solvent or water/alcohol/organic solvent wherein the ratio of water:alcohol is from 20:80 to 80:20 or wherein the ratio of water:alcohol:organic solvent is from 5:80:15 to 80:15:5,
b) mixing the saturated solution of step a) with a metal oxide precursor, a metal oxide or a combination thereof,
c) adding an alcoholic or hydro-alcoholic solution of a polycondensation catalyst to step a) and/or step b),
d) stirring the mixture of steps a) to c) for 2 to 48 h, wherein the carbon donor and the metal oxide precursor, the metal oxide or a combination thereof form a particle,
e) optionally repeating steps a) to c) to form two or more layers of the particle and
f) isolating the formed particles, optionally comprising a pore,
wherein all the steps are performed at a temperature in a range of about −20° C. to about 65° C., preferably in a range of about −5° C. to about 25° C.,
d) optionally additives are added in step a), b) or c).

2. The method according to claim 1, wherein the carbon donor is selected from the group consisting of a monosaccharide, disaccharide, oligosaccharide, polysaccharide, polyol or a combination thereof.

3. The method according to claim 1, wherein the metal oxide precursor is tetraethyoxysilane (TEOS), tetra-methyl-ortho-silicate (TMOS) and/or a metal oxide selected from the group consisting of an oxide of Si, Ti, Fe, Au, Ag, Al, Cu, Cr, Gd, Zn, Zr, Ru, Rh, Pd, Sn, Cd, Sb, Te, U, Er, Yb, or a combination thereof.

4. The method according to claim 1, wherein polycondensation catalyst is a basic polycondensation catalyst for example selected from the group consisting of NaOH, KOH, NH₄OH, LiOH, Mg(OH)₂, a basic amino acid, a basic peptide, N,N′-dimethylethylenediamine or a combination thereof.

5. The method according to claims 2, wherein the amount of the polycondensation catalyst is increased by a factor of about 2× to 10× for increasing the number of pores of the particle.

6. The method according to claim 1, wherein an active agent is added to step a) and/or step b) and the active agent is in pure form, solid or liquid, dissolved in an hydro-alcoholic solution, dissolved in a water-organic solvent or a combination thereof for incorporating the active agent in the polymer or particle for example in the pore.

7. A polymer or particle obtainable by a method according to claim 2.

8. A polymer or particle according to claim 7 comprising a carbon donor such as a carbohydrate, a metal oxide precursor, a metal oxide or a combination thereof, and a polycondensation catalyst, and optionally an active agent, wherein the metal oxide precursor, the metal oxide or the combination thereof forms a scaffold which is covalently connected with carbon of the carbon donor for example wherein 30% to 99% of the scaffold are connected to carbon.

9. The method according to claim 6, wherein the active agent is a peptide or protein, enzyme, DNA, RNA, mRNA, siRNA, miRNA, snoRNA, an oligonucleotide, a small molecule or a combination thereof.

10. A composition comprising a polymer or particle according to claim 7 and a pharmaceutically acceptable excipient, a cosmetic acceptable excipient, an agricultural acceptable excipient or a combination thereof.

11. A medicament comprising the polymer or particle according to claim 7.

12. A method of preventing and/or treating a metabolic disorder/disease such as hyperlipidemia, hypercholesterolemia, hyperglyceridemia, hyperglycemia, insulin resistance, obesity, hepatic steatosis, kidney disease, fatty liver disease, non-alcoholic steatohepatitis, a respiratory disease, an inflammatory disease, obesity, a viral disease, a neuronal disease, a cancer disease, a disease of the central nervous system, a cardio-vascular disease or a combination thereof comprising administering the polymer or particle of claim 7 to a subject in need thereof.

13. A film comprising a polymer or particle according to claim 7.

14. The film according to claim 13, wherein the polymer or particle is dispersed in the film or located on top of one or both sides of the film.

15. A method of administering the polymer or particle of claim 7 to a subject in need thereof wherein the polymer or particle is administered locally or systemically, orally, sublingually, buccally, intravenously, subcutaneously, intramuscularily, enterally, parenterally, topically, vaginally, rectally, intraocularily or a combination thereof.

16. A film comprising the composition according to claim 10.

17. The film according to claim 16, wherein the composition is dispersed in the film or located on top of one or both sides of the film.

18. A method of administering the polymer or the particle of claim 7 to a subject in need thereof, wherein the polymer or particle is administered locally or systemically, orally, sublingually, buccally, intravenously, subcutaneously, intramuscularily, enterally, parenterally, topically, vaginally, rectally, intraocularily or a combination thereof.

19. A method of administering the composition according to claim 10, wherein the composition is administered locally or systemically, orally, sublingually, buccally, intravenously, subcutaneously, intramuscularily, enterally, parenterally, topically, vaginally, rectally, intraocularily or a combination thereof.

20. A method of administering the film according to claim 13, wherein the film is administered locally or systemically, orally, sublingually, buccally, enterally, topically, vaginally, rectally, or a combination thereof.

21. A method of administering the film according to claim 14, wherein the film is administered locally or systemically, orally, sublingually, buccally, enterally, topically, vaginally, rectally, or a combination thereof.
